(19) United States
(12) Patent Application Publication
(10) Pub. No.: US 2004/0123870 A1
(43) Pub. Date: Jul. 1, 2004

(54) Method and apparatus for limiting the movement of the head

(76) Inventors: Richard E. Stamper, Terre Haute, IN (US); G. Joshua Karnes, Terre Haute, IN (US); Keith Moench, Avon, IN (US); David Radue, Martinsville, IN (US); Bradley A. Woodcox, Terre Haute, IN (US)

Correspondence Address: Intellectual Property Group, Bose McKinney & Evans LLP, 2700 First Indiana Plaza, 135 North Pennsylvania Street, Indianapolis in, IN, 46204, US

(21) Appl. No.: 10/633,854

(22) Filed: Aug. 4, 2003

Related U.S. Application Data
(60) Provisional application No. 60/400,541, filed on Aug. 2, 2002.
(60) Provisional application No. 60/402,850, filed on Aug. 12, 2002.

(51) Int. Cl. A61B 19/00

(52) U.S. Cl. 128/869

(57) ABSTRACT

An apparatus and method for restraining the movement of the head of a person. An apparatus and method for restraining the movement of the head of a person relative to a torso of the person. An apparatus and method for simultaneously engaging a plurality of constraints with the head of the person.

### RELATED APPLICATIONS

This application claims the benefit of U.S. Provisional Patent Application Serial No. 60/400,541, filed Aug. 2, 2002, and U.S. Provisional Patent Application Serial No. 60/402,850, filed Aug. 12, 2002, the disclosures of which are each expressly incorporated by reference herein.

### NOTICE

This invention was made with government support under grant reference number 1 R03 AR049424-01 A1 awarded by National Institute of Arthritis and Musculoskeletal and Skin Diseases. The Government has certain rights in the invention.

### BACKGROUND AND SUMMARY OF THE INVENTION

The present invention relates to methods and apparatus for limiting the movement of the head of a person and in particular to methods and apparatus for limiting the movement of the head of a person relative to the torso of the person.

Traditional restraint systems, such as a halo orthosis, minimize motion of the cervical spine after traumatic injury or in recovery after surgery. To immobilize the cervical spine, the halo orthosis provides a rigid structure that fixes the head of the person relative to the person's chest. A halo orthosis typically includes a halo ring, a supporting structure, and a vest. The halo ring is secured to the head of the person through a plurality of halo pins or constraints. The vest is secured to the body of the person, typically the chest and back. The supporting structure couples the vest and the halo ring together such that the movement of the head of the person independent of the torso of the body is limited or greatly restricted. A halo orthosis limits the movement of the head of the person relative to the torso of the person in all six degrees of freedom, three translational degrees of freedom and three rotation degrees of freedom.

Typically, a halo orthosis includes multiple constraints that must be engaged with the head of the person. To ensure that the head is properly fixed, the halo pins typically must pierce through the skin and engage the head by either resting against or embedding in the skull. In practice, it is desirable to evenly balance the forces applied to the head by each pin. As such, each pin should exert the same amount of force to the head as the remaining pins.

Traditionally, an orthopedic surgeon manually balances the force each pin applies to the head through an incremental process wherein each pin is tightened in small increments and in a balanced fashion until the correct forces are evenly applied by all the pins. As such, each pin is tightened individually and it is up to the skill of the orthopedic surgeon to evenly balance the forces applied by each pin.

Over time, one or more of the pins of a halo will typically loosen. This loosening is the result of changes in the geometry of the head and not typically due to the movement of the pin relative to the ring. Changes in the geometry of the head include bone remodeling at the pin sites, a radial recession of the skull at a pin site, changes in the elasticity of the head or skull, and/or other physiological changes in the head or skull geometry which typically cause the pins to loosen over time.

Many effects are the result of pin loosening including the loss of fixation of the head of the person relative to the torso, headaches, and infections. In order to minimize these effects orthopedic surgeons typically tighten each pin well beyond the force needed for securing the head (“pin pre-loading”) in order to delay the onset of the time that the respective pin will lead to a loss of fixation of the head or other effects. However, this pre-loading subjects the head of the person to forces greater than required to secure the head relative to the torso.

Further, proactive and/or corrective pin adjustments to correct pin loosening require undesirably complex and costly follow-up care by highly skilled medical professionals (typically orthopedic surgeons). For the person, pin loosening can cause significant pain, potential loss of immobilization or fixation of the head relative to the torso, and an increased risk of infection.

Thus, there is a need for a restraint system that reduces the likelihood of pin loosening. Additionally, there is a need for a restraint system that requires less time and skill to assemble to the head.

The present invention relates to restraint systems to restrain the movement of the head or to restrain the movement of the head relative to the torso and methods for assembling the restraint systems to the head. Additionally, the present invention relates to restraint systems wherein all of the constraints or pins of the restraint system are simultaneously loaded to provide a balanced restraint system without the need for an extensive incremental tightening process. Further, the present invention relates to restraint systems that can adapt to changes in the skull geometry over time once the restraint system is assembled to the head.

In one exemplary embodiment, an apparatus for limiting the movement of the head of a person, the head having a left half and a right half either or both of which are susceptible to changes in geometry over time is provided. The apparatus comprising a link system including a plurality of links; a first plurality of constraints rigidly coupled to a first link of the link system and adapted to engage the left half of the head of the person; and a second plurality of constraints rigidly coupled to a second link of the link system and adapted to engage the right half of the head of the person. The link system is configured to exert a force on the head of the person through the first plurality of constraints and the second plurality of constraints such that the head is generally fixed. The link system is further configured simultaneously to adapt to changes in the geometry of the head such that the head remains generally fixed over a period of time.

In one example, the link system comprises a first link configured to support the first plurality of constraints; a second link configured to support the second plurality of constraints; a third link coupled to the first link; and a fourth link coupled to the third link and the second link. In another example, the link system comprises a first link configured to support the first plurality of constraints; a second link configured to support the second plurality of constraints; a third link coupled to the first link; and a fourth link coupled to the third link and the second link and the first link is coupled to the third link at a first joint, the first joint configured to constrain the first link to move in a single degree of freedom relative to the third link and the second link is coupled to the fourth link at a second joint, the second joint configured to constrain the second link to move in a single degree of freedom relative to the fourth link. In one variation, the fourth link is a compliant link and is configured to provide a sufficient amount of force to engage the first plurality of constraints and the second plurality of constraints with the head of the person. In another variation, the fourth link is coupled to the third link at a third joint, the third joint configured to constrain the third link to move in a single degree of freedom relative to the fourth link. In yet another variation, the fourth link is coupled to the third link at a third joint, the third joint configured to constrain the third link to move in a single degree of freedom relative to the fourth link and the apparatus further comprises a compliant link coupled to the third link and the fourth link, wherein the compliant link is configured to provide a sufficient amount of force to engage the first plurality of constraints and the second plurality of constraints with the head of the person. In a further variation, the fourth link is coupled to the third link at a third joint, the third joint configured to constrain the third link to move in a single degree of freedom relative to the fourth link and the apparatus further comprises a compliant member coupled to the third link and the fourth link, the compliant member including a compliant link and a force applier.

In another exemplary embodiment, an apparatus for limiting the movement of a head of a person is provided. The apparatus comprising a first link configured to support a first plurality of constraints rigidly coupled to the first link and adapted to engage the left half of the head of the person; a second link configured to support a second plurality of constraints rigidly coupled to the second link and adapted to engage the left half of the head of the person; a third link coupled to the first link at a first joint; a fourth link coupled to the second link at a second joint and coupled to the third link at a third joint; and a force applier coupled to the third link and the fourth link. The force applier configured to load each of the first plurality of constraints and each of the second plurality of constraints simultaneously such that each of the first plurality of constraints and each of the second plurality of constraints engages the head with generally the same amount force.

In an exemplary method, a method of limiting the movement of a head of a person over time, the head being susceptible to changes in geometry over time, is provided. The method comprising the steps of placing a first apparatus adjacent the head of the person, the apparatus including at least a first constraint and a second constraint located adjacent a first side of the head and a third constraint and a fourth constraint located adjacent a second side of the head, engaging each of the first, second, third, and fourth constraints with the head of the person with a force sufficient to limit the movement of the head of the person, and automatically adapting the apparatus to changes in the geometry of the head over such that the head remains generally fixed over time.

In one example, each of the first, second, third, and fourth constraints are engaged simultaneously. In another example, the method further comprises the steps of placing a second apparatus adjacent a torso of the person, the second apparatus being secured to the torso and coupling the first apparatus to the second apparatus such that head of the person is coupled to the torso of the person.

### BRIEF DESCRIPTION OF THE DRAWINGS

FIG. 1 is a diagrammatic view of an exemplary restraint system including a head restraint member for engaging the head or skull of a person and a body restraint member for engaging the body or torso of the person such that the movement of the head of the person is limited relative to the body of the person;

FIG. 2 is an exemplary body restraint member for use with the exemplary restraint system of FIG. 1, the body restraint member including a vest to be secured to the torso of a person and a supporting structure to couple the vest to a head restraint system;

FIG. 3 is another exemplary supporting structure for use with the exemplary body restraint of FIG. 2;

FIG. 4 is an exemplary embodiment of a kinematic structure for the head restraint member of the restraint system of FIG. 1;

FIG. 5 is an exploded, perspective view of an exemplary head restraint member having the kinematic structure of the head restraint member of FIG. 4;

FIG. 6 is a perspective view of the head restraint member of FIG. 5;

FIG. 7 is a cross-sectional view of the head restraint member of FIG. 6 taken along lines 7-7 in FIG. 6;

FIG. 8 is a partial cross-sectional view of the head restraint member of FIG. 6 taken along lines 8-8 in FIG. 6;

FIG. 9 is a variation of the head restraint member of FIG. 6;

FIG. 10 is an exploded, perspective view of another exemplary head restraint member having the kinematic structure of the head restraint member of FIG. 4;

FIG. 11 is a perspective view of the head restraint member of FIG. 5;

FIG. 12 is a rear view of the head restraint member of FIG. 10 with the compliant link removed;

FIG. 13 is a bottom view of the head restraint member of FIG. 10;

FIG. 14 is another exemplary embodiment of a kinematic structure for the head restraint member of the restraint system of FIG. 1;

FIG. 15 is a perspective view of an exemplary head restraint member having the kinematic structure of the head restraint member of FIG. 14;

FIG. 16 is a front view of the head restraint member of FIG. 15;

FIG. 17 is a cross-sectional view of the head restraint member of FIG. 15 taken along lines 17-17 in FIG. 16;

FIG. 18 is a bottom view of the head restraint member of FIG. 15;

FIG. 19 is a diagrammatic view of another exemplary restraint system including a head restraint member for engaging the head or skull of a person and a body restraint member for engaging the body or torso of the person such that movement of the head of the person is limited relative to the body of the person, the head restraint member including a force actuator for simultaneously loading each of the plurality of constraints;

FIG. 20 is a perspective view of an exemplary head restraint member for use with the exemplary restraint system of FIG. 19;

FIG. 21 is another exemplary embodiment of a kinematic structure for the head restraint member of the restraint system of FIG. 1;

FIG. 22 is perspective view of an exemplary head restraint member having the kinematic structure of the head restraint member of FIG. 21;

FIG. 23 is a rear perspective view of the head restraint member of FIG. 22;

FIG. 24 is a front view of the head restraint member of FIG. 22;

FIG. 25 is a bottom view of the head restraint member of FIG. 22;

FIG. 26 is a cross-sectional view of the head restraint member of FIG. 25 taken along lines 26-26 in FIG. 25;

FIG. 27 is an exploded, isometric view of an exemplary restraint system including combination head restraint member and superstructure;

FIG. 28 is an perspective, assembled view the restraint system of FIG. 27;

FIG. 29 is a perspective, assembled view of an exemplary head restraint system;

FIG. 30 is a perspective, exploded view of the restraint system of FIG. 29;

FIG. 31 is a front view of the restraint system of FIG. 29; and

FIG. 32 is a detail cross-sectional view of the force actuator of the exemplary restraint of FIG. 29 taken along lines 32-32 in FIG. 29.

### DETAILED DESCRIPTION

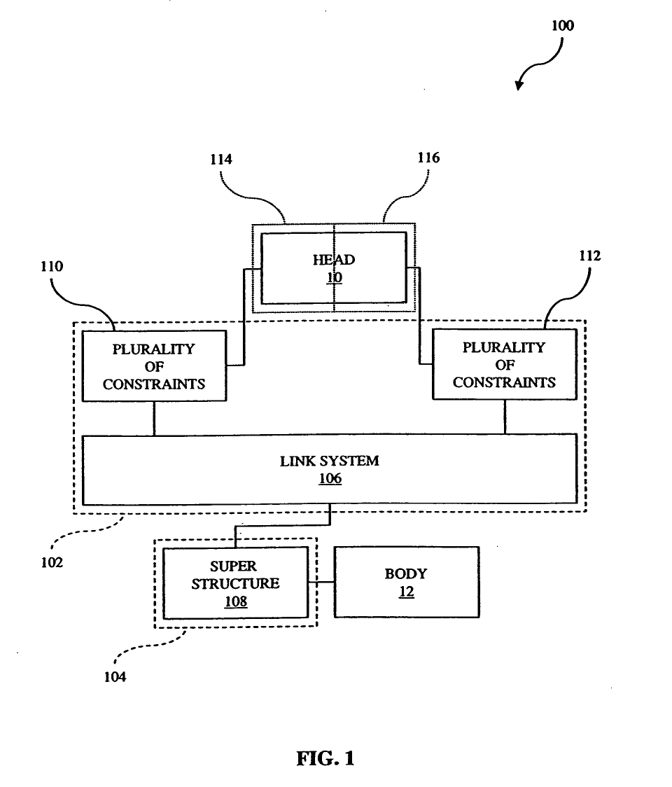

Referring to FIG. 1, a restraint system or apparatus 100 is shown. Restraint system 100 includes a head restraint member 102 for engaging a head or skull 10 of a person and a body restraint member 104 for securing to or engaging the body 12 of the person. In a preferred example, body restraint member 104 is secured to the torso of the person. Head restraint member 102 and body restraint member 104 are coupled together to limit the movement of head 10 relative to body or torso 12. In the illustrated embodiment, a link system 106 of head restraint member 102 is rigidly coupled to a superstructure 108 of body restraint member 104.

As stated above, head restraint member 102 includes link system 106. Link system 106 is coupled to a first plurality of constraints 110 and a second plurality of constraints 112. In one embodiment, first plurality of constraints 110 are located such that the first plurality of constraints 110 engage head 10 generally along a first half 114 of head 10. Second plurality of constraints 112 are located such that the second plurality of constraints 112 engage head 10 generally along a second half 116 of head 10. In general, first half 114 of head 10 is a left half of the head and second half 116 of head 10 is a right half of head 10. First half 114 and second half 116 are generally divided by the sagittal plane of the head. In alternative embodiments, first plurality of constraints 110 and second plurality of constraints 112 engage head 10 in respective first and second regions, not necessarily halves of the head.

Link system 106 exerts a force on head 10 of the person through first plurality of constraints 110 and second plurality of constraints 112 such that head 10 is generally fixed relative to head restraint member 102. It should be understood that if head 10 is fixed relative to head restraint member 102, then head 10 is fixed relative to torso 12 due to body restraint member 104 being secured to torso 12 and body restraint member 104 being rigidly coupled to head restraint member 102.

Link system 106, in a preferred embodiment, is an adaptive link system which is defined as a system of a plurality of links which is capable of automatically adapting to small changes in the geometry of head 10 once head restraint member 102 is applied to head 10 such that head 10 remains generally fixed relative to head restraint member 102. In one example, head restraint member 102 is to be placed on head 10 for a period of eight to twelve weeks. As such, in this example link system 106 is capable of adapting to small changes in the geometry of head 10 during the period of eight to twelve weeks such that head 10 remains generally fixed relative to head restraint member 102.

In a preferred embodiment of head restraint member 102, first plurality of constraints 110 and second plurality of constraints 112 are comprised of pins. Each pin includes a pin tip configured to pierce the skin on the head of the person and embed into the skull of the person. Exemplary pins include pins sold in combination with the Generation 80 cervical product available from Jerome Medical located at 305 Harper Drive, Moorestown, N.J. 08057-3239, pins and pin mounts disclosed in US Published Application No. 20020151831A1 to Stamper et al., filed Feb. 2, 2001, the disclosure of which is incorporated by reference herein, and the pins disclosed in paper titled “Structural Behavior of the Halo Orthosis Pin-Bone Interface: Biomechanical Evaluation of Standard and Newly Designed Stainless Steel Halo Fixation Pins,” by Garfin, S., et al., published in Spine, Vol. 11, No. 10, 1986, the disclosure of which is incorporated by reference herein.

It is further preferred that each pin or constraint 110, 112 be rigidly coupled to the link system 106 such that each pin or constraint 110, 112 does not move relative to link system 106 during the time head restraint member 102 is assembled to head 10. In one example, constraints 110, 112 are permanently rigidly coupled to link system 106 of head restraint member 102 by welding, gluing, epoxying, mechanical fastening, or other suitable means for permanently rigidly coupling constraints 110, 112 to head restraint member 102. In one variation, constraints 110, 112 and the respective portions of link system 106 are integrally formed or made as a single component. In another example, each pin or constraint 110, 112 is moveably coupled to link system 106 during the application of head restraint member 102 to head 10 and rigidly coupled to link system 106 such that the pin or constraint 110, 112 does not move relative to link system 106 once head restraint member 102 is applied to head 10.

Head restraint member 102 when applied to head 10 should exert sufficient force to limit the movement of head 10 relative to head restraint member 102. In one example a force of at least about 30 pounds should be exerted by head restraint member 102. In another example, a force of at least about 40 pounds should be exerted by head restraint member 102. In a further example, a force of at least about 50 pounds should be exerted by head restraint member 102. In yet a further example, a force of at least about 60 pounds should be exerted by head restraint member 102. In yet another example, a force of between about 30 pounds to about 100 pounds should be executed by head restraint member 102. In still another example, a force of between about 40 pounds to about 100 pounds should be executed by head restraint member 102. In yet still another example, a force of between about 50 pounds to about 100 pounds should be executed by head restraint member 102. In still a further example, a force of between about 50 pounds to about 60 pounds should be executed by head restraint member 102.

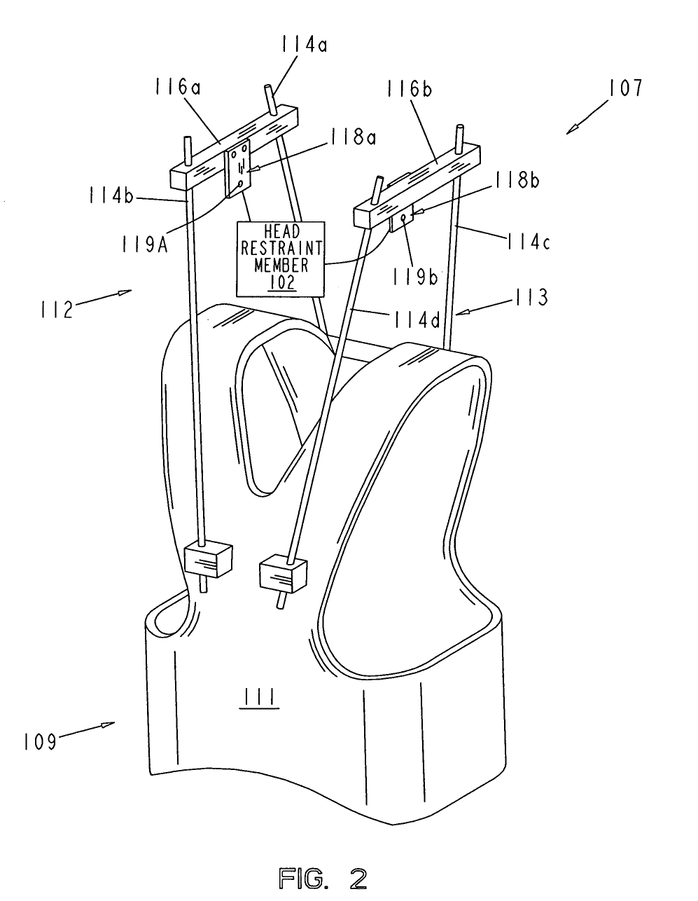

Referring to FIG. 2, an exemplary embodiment 107 of superstructure 108 is shown. Superstructure 107 includes a first portion 109 which is sized and shaped to be secured to torso 12 (see FIG. 1) of the person and a second portion 112 which couples to first portion 109 and to head restraint member 102. In the illustrated example, first portion 109 includes a vest 111 and second portion 112 includes a support structure 113 which includes a plurality of rods 114a, 114b, 114c, 114d coupled to and generally extending upward from vest 111 and cross rods 116a, 116b coupled to respective pairs of rods 114. Cross rod 116a is coupled to rods 114a and 114b. Cross rod 116b is coupled to rods 114c and 114d.

Cross rods 116a and 116b each include a coupling 118a and 118b configured to couple head restraint member 102 to respective cross rods 116a and 116b. In one example, couplings 118a and 118b include a threaded aperture 119a, 119b which receives a fastener. In another example, couplings 118a, 118b are clamps, slides, or other suitable couplings that are capable of being coupled to head restraint member 102 and respective cross rods 116a and 116b, either directly or through one or more fasteners or links.

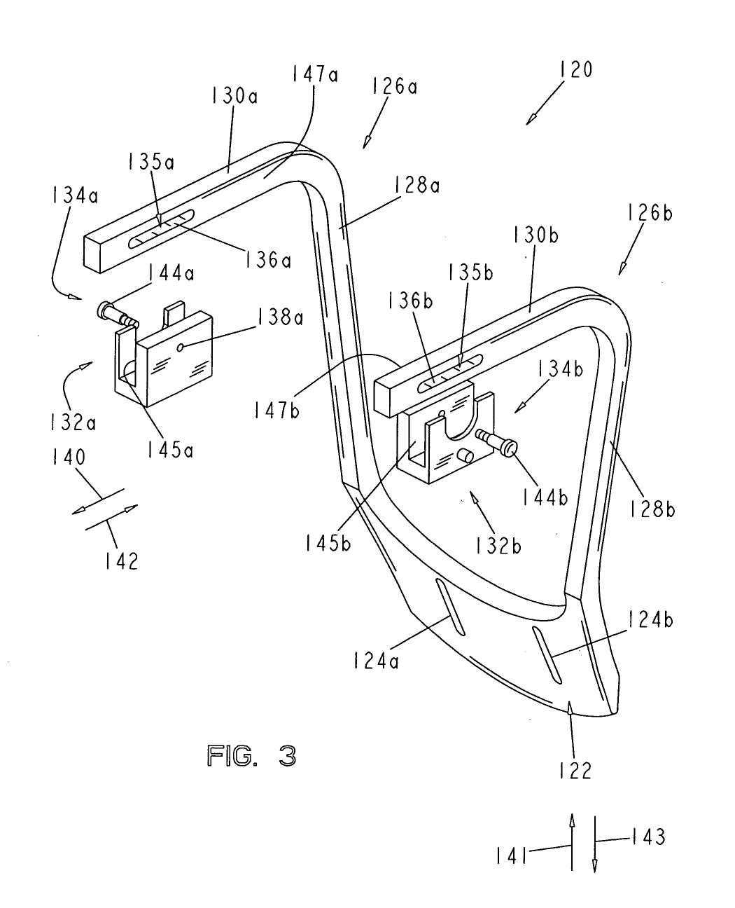

Referring to FIG. 3, a second exemplary support structure 120 for use with first portion 109 is shown. Support structure 120 includes a base member 122 having a plurality of apertures 124a, 124b sized to receive fasteners such that base member 122 may be coupled to vest 111 shown in FIG. 2 with fasteners. Support structure 120 further includes a pair of support arms 126a, 126b. Each of support arms 126a, 126b includes a first portion 128a, 128b extending generally upward relative to base member 122 and a second portion 130a, 130b generally angled relative to first portions 128a, 128b. In one example, second portions 130a, 130b are generally perpendicular to first portions 128a, 128b. In alternative examples second portions 130a, 103b of support structure 120 are angled relative to first portions 128a, 128b of support structure 120 such that an acute angle is made between first portions 128a, 128b and second portions 130a, 130b or such that an obtuse angle is made between first portions 128a, 128b and second portions 130a, 130b.

Support structure 120 further includes couplings 132a, 132b for coupling head restraint member 102, shown in FIG. 1, to support arms 126a, 126b. Couplings 132a, 132b are slidably coupled to the respective support arms 126a, 126b by fasteners 134a, 134b which are received in an elongated slot 135a, 135b of the respective support arms 126a, 126b. Fasteners 134a, 134b slide upon a surface 136a, 136b of the respective slots 135a, 135b and are threadably received into threaded apertures 138, such as threaded aperture 138a in coupling 132a, of the respective couplings 132a, 132b. As such, couplings 132a, 132b are generally restrained to move in directions 140 and 142 relative to support arms 126a, 126b. This movement allows for the positioning of head restraint member 102 to be adjusted relative to support arms 126a, 126b. Similarly apertures 124a, 124b in base member 122 are elongated such that the position of support structure 120 and hence head restraint member 102 is adjustable relative to the vest generally in directions 141 and 143.

Once couplings 132a, 132b are positioned such that head restraint member 102 is properly located relative to support 126a, 126b, i.e. head 10 is properly aligned with body 12, fasteners 134a, 134b are further threaded into apertures 138 such that a head 144a, 144b of respective fasteners 134a, 134b presses against respective support arms 126a, 126b and such that respective couplings 132a, 132b are rigidly coupled to respective support arms 126a, 126b.

In one embodiment, coupling 132a, 132b are rigidly coupled to support arms 126a, 126b at any one of an infinite number of locations permitted by respective elongated slot 135a, 135b. In one example, couplings 132a, 132b are rigidly coupled to the respective support arms 126a, 126b due to the contact of a surface 145a, 145b of respective couplings 132a, 132b and a surface 147a, 147b of the respective support arm 126a, 126b. In another, example, surface 145a, 145b of respective couplings 132a, 132b and surface 147a, 147b of the respective support arm 126a, 126b includes a rough surface such that surfaces 145a, 145b, 147a, 147b mesh to increase the coupling force between couplings 132a, 132b and respective support arms 126a, 126b.

In another embodiment, couplings 132a, 132b are rigidly coupled to respective support arms 126a, 126b at discrete locations. In one example, surfaces 145a, 145b, 147a, 147b include indentations which mate together at discrete intervals. Other exemplary superstructures 108 include superstructures available from Jerome Medical located at 305 Harper Drive, Moorestown, N.J. 08057-3239, including Model No. 545100M.

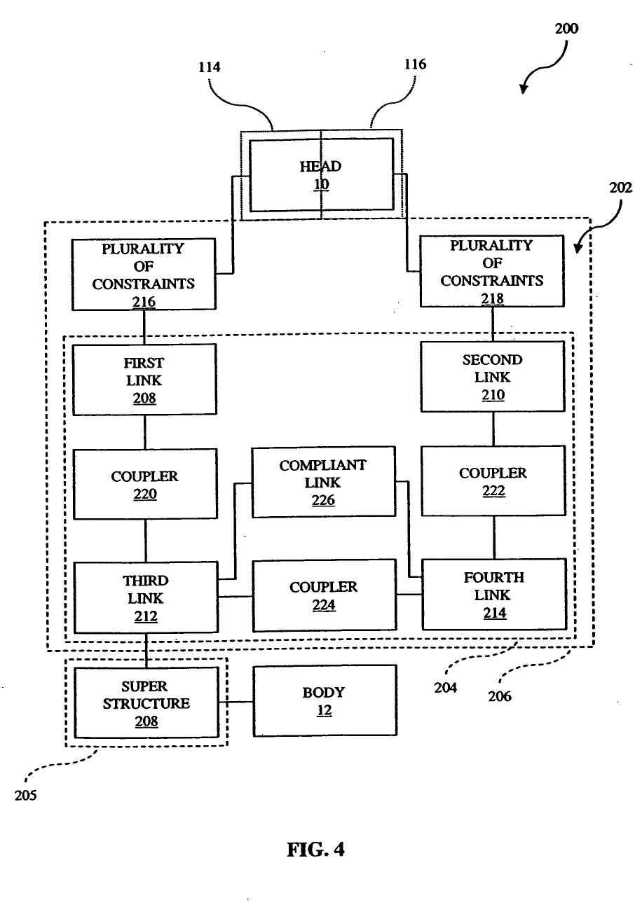

Referring to FIG. 4, an exemplary embodiment 200 of a restraint system according to the present invention is shown. Restraint system 200 includes a head restraint member 206 and a body restraint member 205. Head restraint member 206 includes an adaptive link system 204 and a first plurality of constraints 216 and a second plurality of constraints 218. As discussed above in connection with FIG. 1, an adaptive link system, such as adaptive link system 204, is defined as a system of a plurality of links which is capable of automatically adapting to small changes in the geometry of head 10 once head restraint member 206 is assembled to head 10 such that head 10 remains generally fixed relative to head restraint member 206. Restraint system 200 illustrates a first exemplary kinematic structure 202 for adaptive link system 204.

Link system 204 of head restraint member 206 includes a first link 208, a second link 210, a third link 212 and a four link 214. First link 208 and second link 210 are each capable of supporting plurality of constraints 216, 218, respectively. Preferably, constraints 216, 218 are pins which are rigidly secured to the respective link of first link 208 and second link 210 such that the pins or constraints 216, 218 do not move relative to link system 204 once head restraint member 206 is applied to head 10. In one example, constraints 216, 218 are permanently rigidly coupled to link system 204 of head restraint member 206 by welding, gluing, epoxying, mechanical fastening, or other suitable means for permanently rigidly coupling constraints 216, 218 to head restraint member 206. In one variation, constraints 216, 218 and the respective portions of link system 204, such as first link 208 and second link 210 respectively, are integrally formed or made as a single component.

In another example, each pin or constraint 216, 218 is moveably coupled to link system 204 during the application of head restraint member 206 to head 10 and rigidly coupled to link system 204 such that the pin or constraint 216, 218 does not move relative to link system 204 once head restraint member 206 is assembled to head 10. For example, pins 216, 218 may be threadably received in respective apertures of first link 208 and second link 210 such that pins 216, 218 are moveable relative to link system 204. Pins 216, 218 are then rigidly coupled to link system 204 with a lock nut (not shown).

First link 208 is further coupled to third link 212 by a coupler 220 forming a joint between first link 208 and third link 212. Second link 210 is further coupled to fourth link 214 by a coupler 222 forming a joint between second link 210 and fourth link 214. Preferably, both of couplers 220 and 222 permit generally only a single degree of motion between the respective links of the joint. As such, first link 208 is capable of moving in only one degree of freedom relative to third link 212 and second link 210 is capable of moving in only one degree of freedom relative to fourth link 214. In one example, joints formed by couplers 220 and 222 are revolute joints. In alternative examples, the joints are one of prismatic joints, or slider joints.

Third link 212 is further coupled to fourth link 214 by a coupler 224 forming a joint between third link 212 and fourth link 214. Preferably, coupler 224 permits generally only a single degree of motion between the respective links 212, 214. As such, fourth link 214 is capable of moving in only one degree of freedom relative to third link 212. In one example, the joint found by coupler 224 is a revolute joint. In alternative examples, the joint is one of a pin joint, a prismatic joint, or a slider joint.

Third link 212 and fourth link 214 are further coupled to a compliant link 226. Compliant link 226 exerts a force on links 212, 214 which in turn causes constraints 216, 218 to remain in contact with head 10. In one embodiment, compliant link 226 exerts at least on of a tension force or a compressive force. Compliant link 226 in one example is an elastic band or leaf spring. In another example, compliant link 226 is a spring such as a coil spring. As such, compliant link 226 has a relaxed stated and an unrelaxed state. Compliant link 226 is coupled to third link 212 and fourth link 214 such that when link system 204 is applied to head 10 compliant link 226 is in the unrelaxed state.

Compliant link 226 in combination with the joints formed by couplers 220, 222, and 224 permit link system 204 to adapt to small changes in the geometry of the head. For instance, if the portion of head 10 engaged by one of the plurality of constraints 216 recesses, the force applied by compliant link 226 causes the relative arrangement of at least some of links 208, 210, 212, 214 to adjust such that the one constraint proximate to the recess maintains engagement with head 10. Further, in one example, compliant link 226 and the joints formed by couplers 220, 222, 224 cause the relative arrangement of at least some of links 208, 210, 212, 214 to adjust such that the one constraint proximate to the recess maintains engagement with head 10 and that all constraints exert a generally uniform force against head 10. In a preferred example, compliant link 226 and the joints formed by couplers 220, 222, 224 causes the relative arrangement of at least some of links 208, 210, 212, 214 to adjust such that the one constraint proximate to the recess maintains engagement with head 10, each constraint 216, 218 exerts a generally uniform force against head 10, and each constraint 216, 218 continues to exert generally the same amount of force against head 10 as prior to the recess of head 10 occurring.

Head restraint member 206 when applied to head 10 should exert sufficient force to limit the movement of head 10 relative to head restraint member 206. In one example a force of at least about 30 pounds should be exerted by head restraint member 206. In another example, a force of at least about 40 pounds should be exerted by head restraint member 206. In a further example, a force of at least about 50 pounds should be exerted by head restraint member 206. In yet a further example, a force of at least about 60 pounds should be exerted by head restraint member 206. In yet another example, a force of between about 30 pounds to about 100 pounds should be executed by head restraint member 206. In still another example, a force of between about 40 pounds to about 100 pounds should be executed by head restraint member 206. In yet still another example, a force of between about 50 pounds to about 100 pounds should be executed by head restraint member 206. In still a further example, a force of between about 50 pounds to about 60 pounds should be executed by head restraint member 206.

Referring to FIG. 4, body restraint member of 205 is secured or engaged to body 12 of the person. In a preferred example, body restraint member 205 is secured to the torso of the person. Body restraint member 205 and head restraint member 206 are coupled together to limit the movement of head 10 relative to body or torso 12. In the illustrated embodiment, a superstructure 208 of body restraint member 205 is coupled to link three 512 of link system 204 of head restraint member 206. In one example, the each of the exemplary superstructures described above in connection with restraint system or apparatus 100 may be coupled to head restraint member 206 to couple head 10 to torso 12.

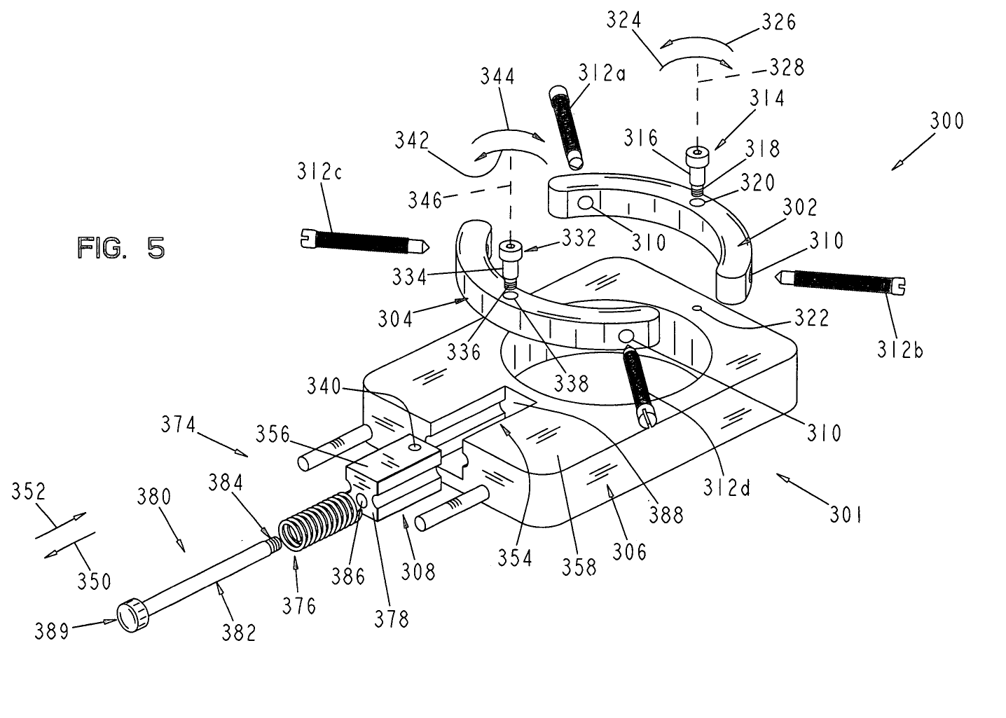

Referring to FIGS. 5-7, an exemplary embodiment 300 of a head restraint system according to the present invention is shown. Head restraint system 300 has the same kinematic structure 202 of head restraint member 206 shown in FIG. 4. Head restraint system 300 includes a link system 301 and a plurality of constraints 312. Link system 301 including a first link 302, a second link 304, a third link 306, and a fourth link 308.

First link 302 and second link 304 each include a plurality of apertures 310 to receive a plurality of constraints or pins 312. First link 302 illustratively includes two apertures 310 to receive pins 312a, 312b, respectively. Second link 304 illustratively includes two apertures 310 to receive pins 312c, 312d, respectively. Although four constraints or pins 312a, 312b, 312c, 312d are illustrated, it is contemplated to have additional constraints 312 and additional apertures 310. Additionally, it is contemplated to have more apertures 310 than constraints 312 such that the location of at least one of constraints 312a, 312b, 312c, 312d may be altered relative to the other of constraints 312a, 312b, 312c, 312d.

In one example, constraints 312a and 312b are generally equidistant from joint 330 and constraints 312c and 312d are generally equidistance from joint 348. In one variation, the distance from joint 330 to each of constraints 312a and 312b is generally equal to the distance from joint 348 to each of constraints 312c and 312d. In another variation, the distance from joint 330 to each of constraints 312a and 312b is generally not equal to the distance from joint 348 to each of constraints 312c and 312d. In another example, at least one pair of constraints 312a and 312b and constraints 312c and 312d are generally non-equidistant from the respective joint 330 and joint 348. In one variation, the distance from joint 330 to each of constraints 312a and 312b is generally equal and the distance from joint 348 to each of constraints 312c and 312d is generally not equal. In another variation, the distance from joint 330 to each of constraints 312a and 312b is generally not equal and the distance from joint 348 to each of constraints 312c and 312d is generally not equal.

Preferably, constraints 312a, 312b, 312c, 312d are pins which are rigidly secured to the respective link of first link 302 and second link 304 such that the pins or constraints 312a, 312b, 312c, 312d do not move relative to the respective link of first link 302 and second link 304 once head restraint member 300 is assembled to head 10. In one example, constraints 312a, 312b, 312c, 312d are permanently rigidly coupled to link system 301 of head restraint member 300 by welding, gluing, epoxying, mechanical fastening, or other suitable means for permanently rigidly coupling constraints 312a, 312b, 312c, 312d to head restraint member 300. In one variation, constraints 312a, 312b, 312c, 312d and the respective link of first link 302 and second link 304 are integrally formed or made as a single component.

In another example, each pin or constraint 312a, 312b, 312c, 312d is moveably coupled to the respective link of first link 302 and second link 304 during the application of head restraint member 300 to head 10 and rigidly coupled to the respective link of first link 302 and second link 304 such that the pin or constraint 312a, 312b, 312c, 312d does not move relative to the respective link of first link 302 and second link 304 once head restraint member 300 is applied to head 10. For example, pins 312a, 312b, 312c, and 312d may be threadably received in respective apertures 310 of first link 302 and second link 304 such that pins 312a, 312b, 312c, and 312d are moveable relative to link system 301. Pins 312a, 312b, 312c, and 312d are then rigidly coupled to link system 301 with a lock nut.

Both of first link 302 and second link 304 include an arcuate longitudinal extent to approximate the curvature of the left half of the head and the right half of the head respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 302 and second link 304 and/or orientation of apertures 310 is such that pins or constraints 312a, 312b, 312c, 312d are generally normal to head 10 when head restraint member 300 is applied to head 10.

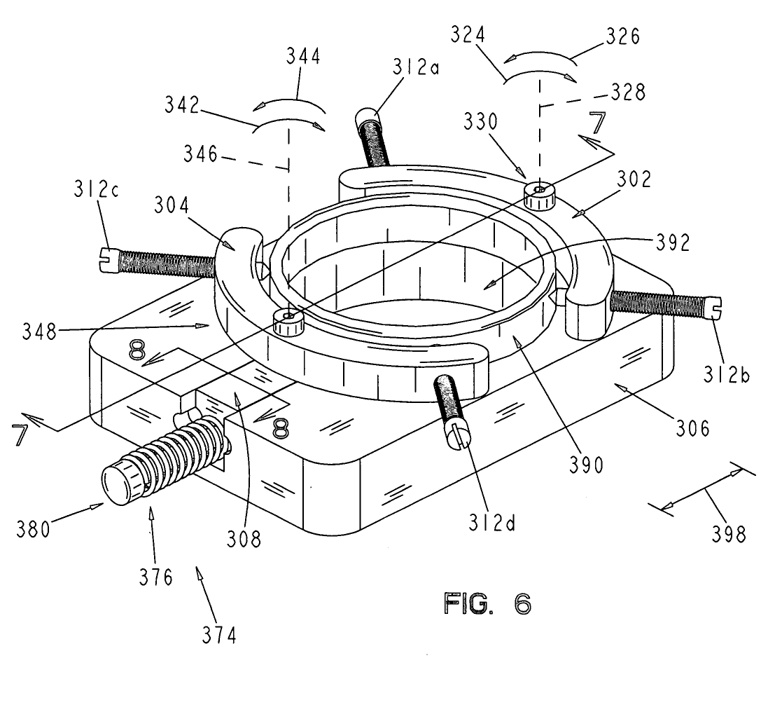

First link 302 is further coupled to third link 306 by a coupler or pin 314. Pin 314 includes a shoulder portion 316 and a threaded portion 318. First link 306 includes an aperture 320 sized to receive the shoulder portion 316 of coupler 314. Third link 306 includes a threaded aperture 322 which is sized to threadably receive threaded portion 318 of coupler 314. Once first link 302 is coupled to third link 306 by coupler 314, first link 302 is constrained to move in a single degree of freedom, namely to rotate in directions 324 and 326 about an axis 328 of coupler 314. As such, coupler 314 creates a revolute joint 330 (see FIG. 6) between first link 302 and third link 306. In alternative examples, the joint between the first link and the third link is one of a prismatic joint or a slider joint.

Second link 304 is further coupled to fourth link 308 by a coupler or pin 332. Pin 332 includes a shoulder portion 334 and a threaded portion 336. Second link 304 includes an aperture 338 sized to receive the shoulder portion 334 of coupler 332. Fourth link 308 includes a threaded aperture 340 which is sized to threadably receive threaded portion 336 of coupler 332. Once second link 304 is coupled to fourth link 308 by coupler 332, second link 304 is constrained to move in a single degree of freedom, namely to rotate in directions 342 and 344 about an axis 346 of coupler 332. As such, coupler 332 creates a revolute joint 348 (see FIG. 6) between second link 304 and fourth link 308. In alternative examples, the joint between the second link and the fourth link is one of a prismatic joint or a slider joint.

Third link 306 and fourth link 308 are coupled together such that fourth link 308 is constrained to move in a single degree of freedom, namely to translate in directions 350 and 352 along a channel 354 formed in third link 306. Channel 354 is sized to receive fourth link 308. In one example, channel 354 is sized such that a top surface 356 of fourth link 308 is generally flush with a top surface 358 of third link 306. In alternative examples, the top surface of the fourth link is offset either above or below the top surface of the third link. Referring to FIG. 8, channel 354 further includes keyways 360a, 360b sized to receive at least a portion of keys 362a, 362b. Fourth link 308 further includes keyways 364a, 364b sized to receive at least a portion of keys 362a, 362b. Keys 362a, 362b interact with keyways 360a, 360b, 364a, 364b to prevent or limit the movement of fourth link 308 relative to third link 306 in directions 366 and 368. In the illustrated embodiment, keys 362a, 362b are generally cylindrical and keyways 360a, 360b, 364a, 364b each include a concave surface 370a, 370b, 372a, 372b sized to receive keys 362a, 362b. In alternative embodiments, the keys and corresponding keyways are rectangular, triangular, hexagonal, elliptical, or other shape.

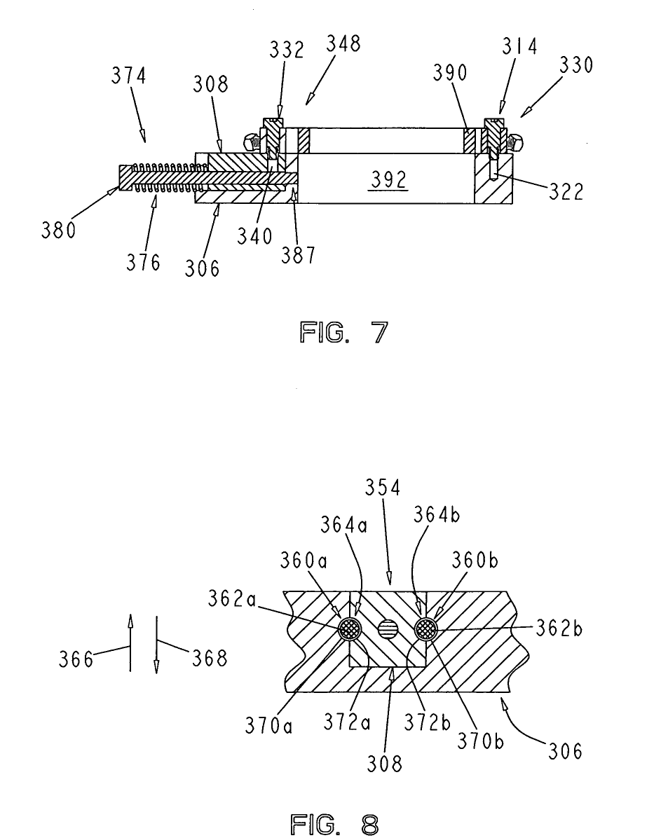

Referring to FIG. 5, although fourth link 308 is able to move in both directions 350 and 352 relative to third link 306, a compliant link 374 biases fourth link 308 in direction 352 relative to third link 306. In one embodiment, compliant link 374 exerts at least on of a tension force or a compressive force on fourth link 308. Compliant link 374 includes a spring 376 which is compressed against a surface 378 of fourth link 308 by a coupler or pin 380. Pin 380 includes a shoulder portion 382 and a threaded portion 384. Fourth link 308 includes a longitudinal aperture 386 sized to receive shoulder portion 382 of pin 380. Third link 306 includes a threaded aperture 387 (see FIG. 7) in surface 388 which is sized to threadably receive threaded portion 384 of pin 380. Once pin 380 has been coupled to third link 306, spring 376 is compressed between surface 378 of fourth link 308 and a head 389 of pin 380 resulting in fourth link 308 being biased in direction 352.

It should be noted that head restraint system 300 is shown in FIGS. 5-8 as a breadboard model to test the operation of head restraint system 300. As shown in FIG. 6, pins 312a, 312b, 312c, 312d engage a ring 390 to simulate the engagement of a head. However, head restraint system 300 may be used to restrain a head, such as head 10, as depicted in FIGS. 5-8, since an opening 392 in third link 306 is sized to receive head 10. However, the shape and thickness of third link 306 should be altered to be generally a ring of constant thickness between an internal surface and an external surface.

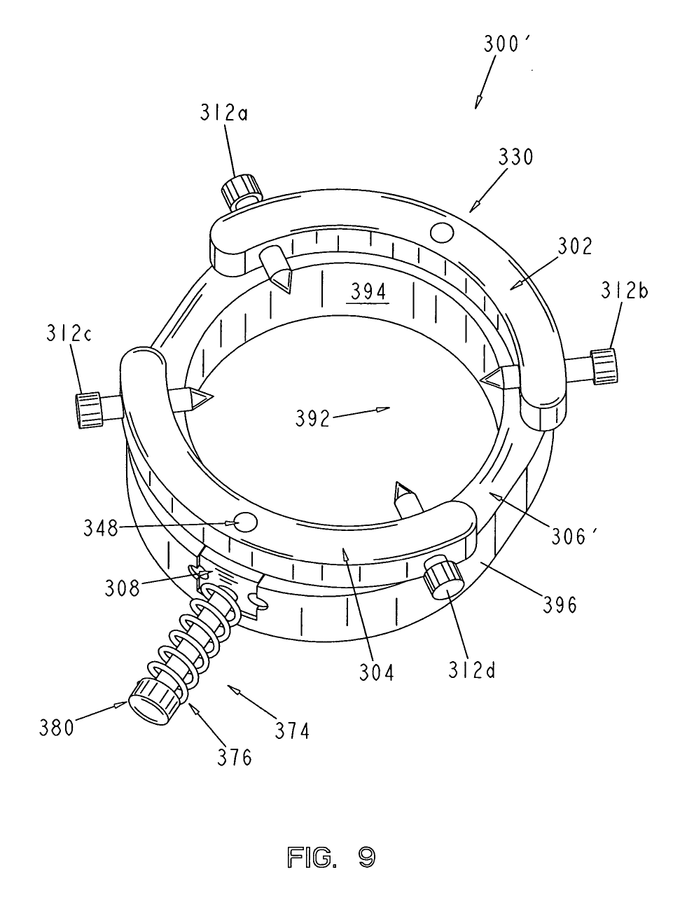

Referring to FIG. 9, a head restraint system 300′ is shown. Head restraint system 300′ is generally similar to head restraint system 300 except that third link 306 of head restraint system 300 is replaced by a third link 306′ that is generally ring shaped and has a generally constant thickness between an internal surface 394 of third link 306′ and an external surface 396 of third link 306′.

Head restraint system 300, 300′ is further coupled to a body restraint system, such as body restraint member 205, shown in FIG. 4 to limit the movement of head 10 relative to body 12. Referring to FIG. 4, body restraint member of 205 is secured or engaged to body 12 of the person. In a preferred example, body restraint member 205 is secured to the torso of the person. Body restraint member 205 and head restraint member 300 may then be coupled together to limit the movement of head 10 relative to body or torso 12. In one example, the each of the exemplary superstructures described above in connection with restraint system or apparatus 100 may be coupled to head restraint member 300 to couple head 10 to torso 12.

Head restraint system 300 is applied to a head of the person as follows. In a first exemplary method, pins 312a, 312b, 312c, 312d are assembled to respective links 302, 304 such that pins 312a, 312b, 312c, 312d extend inward from links 302 and 304 by a predetermined amount. Fourth link 308 is translated generally in direction 350 such that a spacing 398 (see FIG. 6) between first link 302 and second link 304 is increased. The movement of fourth link 308 in direction 350 further causes spring 376 of compliant member 374 to compress. In one example, a spacer tool is used to maintain the orientation of fourth link 308 relative to third link 306 prior to assembly of complaint link 374.

Head restraint member 300 is placed over head 10. Fourth link 308 is gradually allowed to translate in direction 352 such that pins 312a, 312b, 312c, 312d are brought into engagement with head 10. At this point, head restraint member 300 applied to head 10. The applied head restraint member 300 is further assembled to a body restraint device, such as body restraint member 205 to limit the movement of head 10 relative to the torso 12 of the person.

It should be noted that the force exerted by pins 312a, 312b, 312c, 312d is governed by the force exerted by compliant link 374 on third link 306 and fourth link 308. In the illustrated example, the force exerted by pins 312a, 312b, 312c, 312d is governed by a spring constant of spring 376. As such, the orthopedic surgeon may adjust the force exerted by pins 312a, 312b, 312c, 312d by utilizing a spring 376 having a predetermined spring constant. Alternatively, in embodiments where pins 312a, 312b, 312c, 312d are threadably coupled to first link 302 and second link 304, the force exerted by the respective pins 312a, 312b, 312c, 312d may be adjusted by either advancing the respective pin in the respective aperture 310 to increase the force or by retracting the respective pin outward of respective aperture 310 to decrease the force. It should be noted that the advancement or retraction of a single constraint 312a, 312b, 312c, 312d results in a corresponding increase or decrease in force for all of constraints 312a, 312b, 312c, 312d due to the arrangement of links 302, 304, 306, 308 and compliant link 374.

In a second exemplary method for applying head restraint 300 to head 10 complaint link 374 is not initially assembled to the remaining links 302, 304, 306, 308. Initially, head 10 is placed between first link 302 and second link 304 and engaged with pins 312a, 312b, 312c, 312d. The position of fourth link 308 is maintained relative to third link 306. Compliant link 374 is then assembled to bias fourth link 308 in direction 352 relative to third link 306. At this point, head restraint member 300 is applied to head 10. Head restraint 300 is further coupled to a body restraint device, such as body restraint member 205 to limit the movement of head 10 relative to torso 12 of the person. In an example a spacer tool is used to maintain the orientation of fourth link 308 relative to third link 306 prior to assembly of complaint link 374.

Head restraint member 300 when applied to head 10 should exert sufficient force to limit the movement of head 10 relative to head restraint member 300. In one example a force of at least about 30 pounds should be exerted by head restraint member 300. In another example, a force of at least about 40 pounds should be exerted by head restraint member 300. In a further example, a force of at least about 50 pounds should be exerted by head restraint member 300. In yet a further example, a force of at least about 60 pounds should be exerted by head restraint member 300. In yet another example, a force of between about 30 pounds to about 100 pounds should be executed by head restraint member 300. In still another example, a force of between about 40 pounds to about 100 pounds should be executed by head restraint member 300. In yet still another example, a force of between about 50 pounds to about 100 pounds should be executed by head restraint member 300. In still a further example, a force of between about 50 pounds to about 60 pounds should be executed by head restraint member 300.

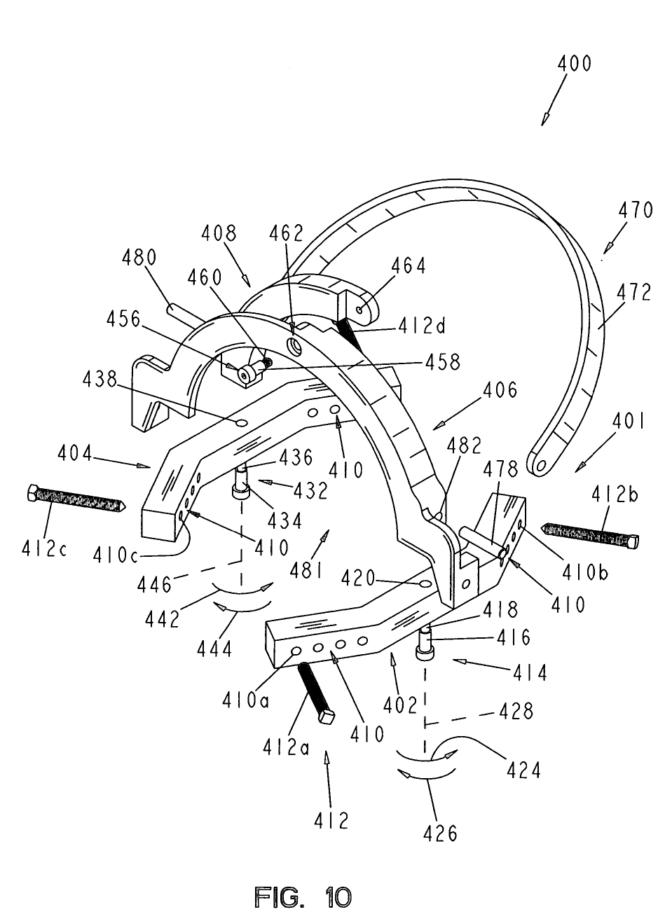

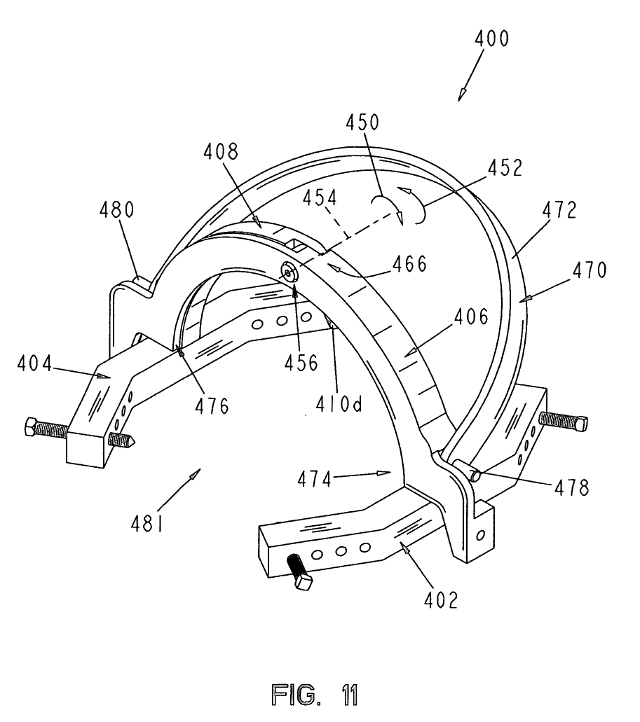

Referring to FIGS. 10-13, an exemplary embodiment 400 of a head restraint system according to the present invention is shown. Head restraint system 400 has the same kinematic structure 202 as head restraint member 206 shown in FIG. 4. Head restraint system 400 includes a link system 401, a first plurality of constraints 516 and a second plurality of constraints 518. Link system 401 includes a first link 402, a second link 404, a third link 406, and a fourth link 408.

First link 402 and second link 404 each include a plurality of apertures 410 to receive a plurality of constraints or pins 412. Referring to FIGS. 10 and 11, first link 402 includes plurality of apertures 410 each configured to receive constraint 412. As shown in FIGS. 10 and 11, first link 402, in one example, includes two constraints 412a and 412b received in apertures 410a, 410b, respectively. It is contemplated that constraints 412a, 412b may be received in other apertures 410 and/or that additional constraints 412 are received in additional apertures 410. Second link 304 includes plurality of apertures 410 each configured to receive constraint 412. As shown in FIGS. 10 and 11, second link 404, in one example, includes two constraints 412c and 412d received in apertures 410c, 410d, respectively. It is contemplated that constraints 410c, 410d may be received in other apertures 410 and/or that additional constraints 412 are received in additional apertures 410.

Preferably, constraints 412a, 412b, 412c, 412d are pins which are rigidly secured to the respective link of first link 402 and second link 404 such that the pins or constraints 412a, 412b, 412c, 412d do not move relative to the respective link of first link 402 and second link 404 once head restraint member 400 is assembled to head 10. In one example, constraints 412a, 412b, 412c, 412d are permanently rigidly coupled to link system 401 of head restraint member 400 by welding, gluing, epoxying, mechanical fastening, or other suitable means for permanently rigidly coupling constraints 412a, 412b, 412c, 412d to head restraint member 400. In one variation, constraints 412a, 412b, 412c, 412d and the respective first link 402 and second link 404 are integrally formed or made as a single component.

In another example, each pin or constraint 412a, 412b, 412c, 412d is moveably coupled to the respective link of first link 402 and second link 404 during the application of head restraint member 400 to head 10 and rigidly coupled to the respective link of first link 402 and second link 404 such that the respective pin or constraint 412a, 412b, 412c, 412d does not move relative to the respective link of first link 402 and second link 404 once head restraint member 400 is applied to head 10. For example, pins 412a, 412b, 412c, 412d may be threadably received in respective apertures of first link 402 and second link 404 such that pins 412a, 412b, 412c, 412d are moveable relative to link system 404. Pins 412a, 412b, 412c, 412d are then rigidly coupled to link system 401 with a lock nut.

Both of first link 402 and second link 404 include a faceted longitudinal extent approximating an arcuate longitudinal extent which in turn is an approximation of the curvature of the left half of the head and the right half of the head, respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 402 and second link 404 and/or orientation of apertures 410 is such that pins or constraints 412a, 412b, 412c, 412d are generally normal to head 10 when head restraint member 400 is applied to head 10.

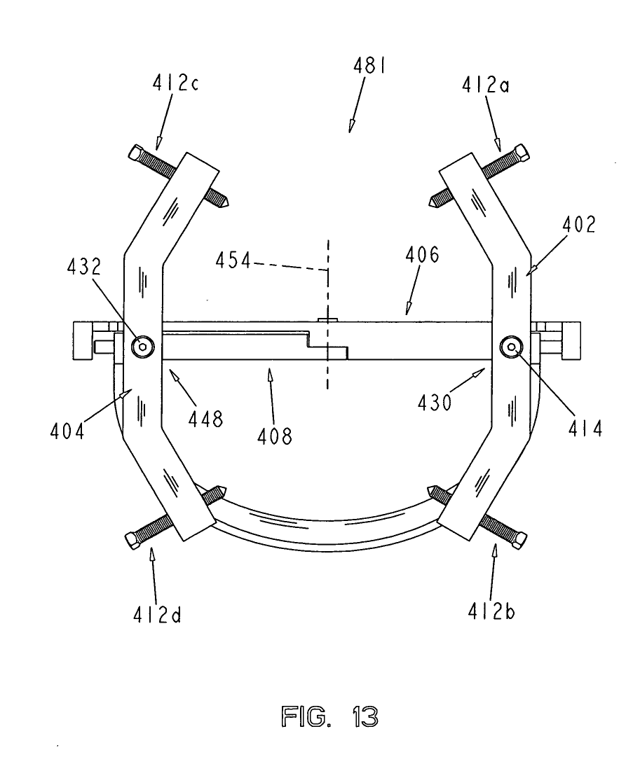

First link 402 is further coupled to third link 406 by a coupler or pin 414. Pin 414 includes a shoulder portion 416 and a threaded portion 418. First link 402 includes an aperture 420 sized to receive shoulder portion 416 of coupler 414. Third link 406 includes a threaded aperture (not shown) which is sized to threadably receive threaded portion 418 of coupler 414. Once first link 402 is coupled to third link 406 by coupler 414, first link 402 is constrained to move in a single degree of freedom, namely to rotate in directions 424 and 426 about an axis 428 of coupler 414. As such, coupler 414 creates a revolute joint 430 (see FIG. 13) between first link 402 and third link 406. In alternative examples, the joint between the first link and the third link is one of a prismatic joint or a slider joint.

Second link 404 is further coupled to fourth link 408 by a coupler or pin 432. Pin 432 includes a shoulder portion 434 and a threaded portion 436. Second link 404 includes an aperture 438 sized to receive the shoulder portion 434 of fastener 432. Fourth link 408 includes a threaded aperture (not shown) which is sized to threadably receive threaded portion 436 of coupler 432. Once second link 404 is coupled to fourth link 408 by coupler 432, second link 404 is constrained to move in a single degree of freedom, namely to rotate in directions 442 and 444 about an axis 446 of coupler 432. As such, coupler 432 creates a revolute joint 448 (see FIG. 13) between second link 404 and fourth link 408. In alternative examples, the joint between the second link and the fourth link is one of a prismatic joint or a slider joint.

Referring to FIG. 11, third link 406 and fourth link 408 are coupled together such that fourth link 408 is constrained to move in a single degree of freedom, namely to rotate in directions 450 and 452 about an axis 454 of a coupler 456 which couples third link 406 and fourth link 408. Referring to FIG. 10, coupler 456 includes a shoulder portion 458 and a threaded portion 460. Third link 406 includes an aperture 462 sized to receive the shoulder portion 458 of coupler 456. Fourth link 408 includes a threaded aperture 464 which is sized to threadably receive threaded portion 460 of coupler 456. Once fourth link 408 is coupled to third link 406 by coupler 456, fourth link 408 is constrained to rotate in directions 450 and 452 (see FIG. 11). As such, coupler 456 creates a revolute joint 466 (see FIG. 11) between third link 406 and fourth link 408. In alternative examples, the joint between the third link and the fourth link is one of a prismatic joint or a slider joint.

Referring to FIG. 11, although fourth link 408 is able to rotate in both directions 450 and 452 relative to third link 406, a compliant link 470 biases fourth link 408 in direction 452 relative to third link 406. In one embodiment, compliant link 470 exerts at least on of a tension force or a compressive force. Compliant link 470 includes an elastic band 472 which is coupled to a first end 474 of third link 406 and a first end 476 of fourth link 408. First end 474 of third link 406 and first end 476 of fourth link 408 each includes a pin or coupler 478, 480, respectively. Pins 478, 480 are received in apertures 482 of third link 406 and fourth link 408, respectively. Elastic band 472 includes a pair of apertures 484, (not shown) which are sized to receive pins 478, 480, respectively. In alternative embodiments, elastic band 472 is coupled to third link 406 and fourth link 408 by one or more of gluing, epoxying, mechanical fastening, or other suitable means. In an alternative embodiment, compliant link 470 is a compression spring coupled to third link 406 and fourth link 408.

Elastic band 472, in one example, is made from a non-magnetic material such that compliant member 470 does not interfere with MRI imaging of head 10. One example non-magnetic material is a glass-filled epoxy available from Composiflex located at 8100 Hawthorne Drive Erie Pa. 16509. In another example, elastic band 472 is made from any material which is capable of exerting a force on third link 406 and fourth link 408.

Compliant link 470, in a preferred embodiment, is not in a relaxed state when head restraint member 400 is applied to head 10. As such, compliant link 470 biases first end 476 of fourth link 408 towards first end 474 of third link 406 such that fourth link 408 is biased to rotate generally in direction 452 relative to third link 406.

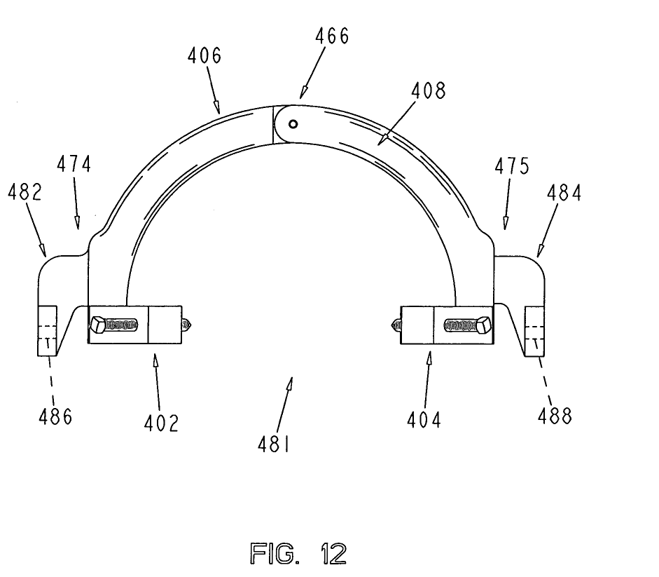

Referring to FIG. 12, third link 406 spans an opening 481 of head restraint member 400 sized to receive a head of the person. Third link 406 further includes a first mounting bracket 482 located at first end 474 and a second mounting bracket 484 located at a second end 475. Mounting brackets 482, 484 respectively include mounting apertures 486 and 488. Mounting apertures 486 and 488 are configured to receive fasteners to couple third link 406 to a body restraint device, such as body restraint member 205 of FIG. 4. As such, third link 406 alone provides the coupling between head restraint member 400 and body restraint member 205. Therefore, the movement of first link 402 relative to third link 406, second link 404 relative to fourth link 408, and fourth link 408 relative to third link 406 is isolated from the coupling of head restraint member 400 to body restraint member 205. In the illustrated embodiment, compliant link 470 spans opening 480 of head restraint member 400 sized to receive a head of the person.

It is contemplated that the placement of constraints 412a, 412b, 412c, 412d may be balanced or unbalanced. In one example, constraints 412a and 412b are generally equidistant from joint 430 and constraints 412c and 412d are generally equidistance from joint 448. In one variation, the distance from joint 430 to each of constraints 412a and 412b is generally equal to the distance from joint 448 to each of constraints 412c and 412d. In another variation, the distance from joint 430 to each of constraints 412a and 412b is generally not equal to the distance from joint 448 to each of constraints 412c and 412d. In another example, at least one pair of constraints 412a and 412b and constraints 412c and 412d are generally non-equidistant from the respective joint 430 and joint 448. In one variation, the distance from joint 430 to each of constraints 412a and 412b is generally equal and the distance from joint 448 to each of constraints 412c and 412d is generally not equal. In another variation, the distance from joint 430 to each of constraints 412a and 412b is generally not equal and the distance from joint 448 to each of constraints 412c and 412d is generally not equal.

Head restraint system 400 is applied to a head of the person as follows. In a first exemplary method, pins 412a, 412b, 412c, 412d are assembled to respective links 402, 404 such that pins 412a, 412b, 412c, 412d extend inward from links 402 and 404 by a predetermined amount. Fourth link 408 is rotated generally in direction 450 such that the spacing of opening 481 between first link 402 and second link 404 is increased. The movement of fourth link 408 in direction 450 further causes elastic band 472 of compliant link 470 to be further stretched. In an example a spacer tool is used to stretch elastic band 472 and to maintain the orientation of fourth link 408 relative to third link 406 prior to applying head restraint member 400 to head 10.

Head restraint member 400 is placed over head 10. Fourth link 408 is gradually allowed to rotate in direction 452 such that pins 412a, 412b, 412c, 412d are brought into engagement with head 10. At this point, head restraint member 400 is applied to head 10. The applied head restraint member 400 is further assembled to a body restraint device, such as body restraint member 205 to limit the movement of head 10 relative to the torso 12 of the person.

It should be noted that the force exerted by pins 412a, 412b, 412c, 412d is governed by the force exerted by compliant link 470 on third link 406 and fourth link 408. In the illustrated example, the force exerted by pins 412a, 412b, 412c, 412d is governed by the characteristics of elastic band 472. As such, the orthopedic surgeon may adjust the force exerted by pins 412a, 412b, 412c, 412d by utilizing an elastic band having certain predetermined characteristics. Alternatively, in embodiments where pins 412a, 412b, 412c, 412d are threadably coupled to first link 402 and second link 404, the force exerted by the respective pins 412a, 412b, 412c, 412d may be adjusted by either advancing the respective pin in the respective aperture 410 to increase the force or by retracting the respective pin outward of respective aperture 410 to decrease the force. It should be noted that the advancement or retraction of a single constraint 412a, 412b, 412c, 412d results in a corresponding increase or decrease in force for all of constraints 412a, 412b, 412c, 412d due to the arrangement of links 402, 404, 406, 408 and compliant link 470.

In a second exemplary method for applying head restraint 400 to head 10 complaint link 470 is not initially assembled to the remaining links 402, 404, 406, 408. Initially, head 10 is placed between first link 402 and second link 404 and engaged with pins 412a, 412b, 412c, 412d. The position of fourth link 408 is maintained relative to third link 406. Compliant link 470 is then assembled to bias fourth link 408 in direction 452 relative to third link 406. At this point, head restraint member 400 is applied to head 10. Head restraint 400 is further coupled to a body restraint device, such as body restraint member 205 to limit the movement of head 10 relative to torso 12 of the person. In an example a spacer tool is used to maintain the orientation of fourth link 408 relative to third link 406 prior to assembly of complaint link 474.

Head restraint member 400 when applied to head 10 should exert sufficient force to limit the movement of head 10 relative to head restraint member 400. In one example a force of at least about 30 pounds should be exerted by head restraint member 400. In another example, a force of at least about 40 pounds should be exerted by head restraint member 400. In a further example, a force of at least about 50 pounds should be exerted by head restraint member 400. In yet a further example, a force of at least about 60 pounds should be exerted by head restraint member 400. In yet another example, a force of between about 30 pounds to about 100 pounds should be executed by head restraint member 400. In still another example, a force of between about 40 pounds to about 100 pounds should be executed by head restraint member 400. In yet still another example, a force of between about 50 pounds to about 100 pounds should be executed by head restraint member 400. In still a further example, a force of between about 50 pounds to about 60 pounds should be executed by head restraint member 400.

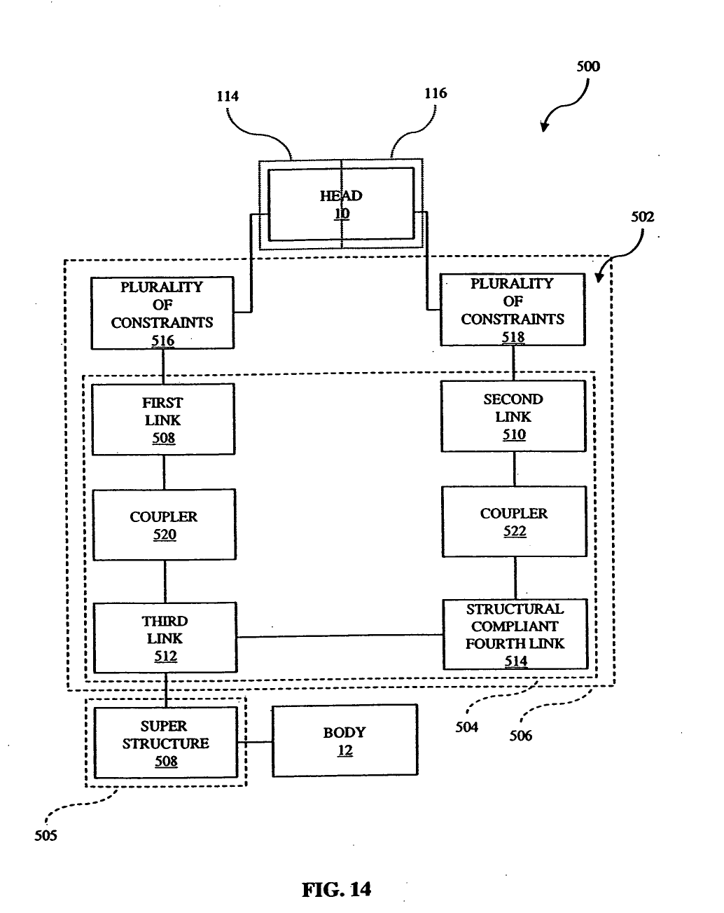

Referring to FIG. 14, an exemplary embodiment 500 of a restraint system according to the present invention is shown. Restraint system 500 includes a head restraint member 506 and a body restraint member 505. Head restraint member 506 includes a link system 504, a first plurality of constraints 516, and a second plurality of constraints 518. As discussed above in connection with FIG. 1, an adaptive link system, such as adaptive link system 504, is defined as a system of a plurality of links which is capable of automatically adapting to small changes in the geometry of head 10 once head restraint member 506 is applied to head 10 such that head 10 remains generally fixed relative to head restraint member 506. Restraint system 500 illustrates another exemplary kinematic structure 502 for an adaptive link system 504.

Link system 504 includes a first link 508, a second link 510, a third link 512 and a four link 514. First link 508 and second link 510 are each capable of supporting a plurality of constraints 516, 518, respectively. Preferably, constraints 516, 518 are pins which are rigidly secured to the respective link of first link 508 and second link 510 such that the pins or constraints 516, 518 do not move relative to link system 504 once head restraint member 506 is assembled to head 10. In one example, constraints 516, 518 are permanently rigidly coupled to link system 504 of head restraint member 506 by welding, gluing, epoxying, mechanical fastening, or other suitable means for permanently rigidly coupling constraints 516, 518 to head restraint member 506. In one variation, constraints 516, 518 and the respective portions of link system 504 are integrally formed or made as a single component.

In another example, each pin or constraint 516, 518 is moveably coupled to the link system 504 during the application of head restraint member 506 to head 10 and rigidly coupled to the link system 504 such that the pin or constraint 516, 518 does not move relative to link system 504 once head restraint member 506 is applied to head 10. For example, pins 516, 518 may be threadably received in respective apertures of first link 508 and second link 510 such that pins 516, 518 are moveable relative to link system 504. Pins 516, 518 are then rigidly coupled to link system 504 with a lock nut.

First link 508 is further coupled to third link 512 through a coupler 520 formerly a joint between first link 508 and third link 512. Second link 510 is further coupled to fourth link 514 through a coupler 522 formerly a joint between first link 508 and third link 512. Preferably, both of couplers 520 and 522 permit generally only a single degree of motion between the respective links of the joint. As such, first link 508 is capable of moving in only one degree of freedom relative to third link 512 and second link 510 is capable of moving in only one degree of freedom relative to fourth link 514. In one example, the joints formed by couplers 520 and 522 are revolute joints. In alternative examples, the joints are one of prismatic joints or slider joints.

Third link 512 is further coupled to fourth link 514. Fourth link 514 preferably is rigidly coupled to third link 512 and is further made of a compliant material such that fourth link either directly or through third link 512 exerts a force on links 508, 510 which in turn causes constraints 516, 518 to remain in contact with head 10. Compliant fourth link 514 in one example is an elastic band or leaf spring. As such, complaint fourth link 514 has a relaxed stated and an unrelaxed state. Compliant fourth link 514 is configured such that when head restraint member 506 is applied to head 10 fourth link 514 is in the unrelaxed state.

In one example, complaint fourth link 514, is made from a non-magnetic material such that complaint member 514 does not interfere with MRI imaging of head 10. One example non-magnetic material is a glass-filled epoxy available from Composiflex located at 8100 Hawthorne Drive Erie Pa. 16509. In another example, fourth link 514 is made from any material which is capable of exerting a force resulting in constraints 516, 518 engaging head 10 over time.

The compliance of fourth link 514 in combination with the joints formed by couplers 520 and 522 permit link system 504 to adapt to small changes in the geometry of the head. For instance, if the portion of head 10 engaged by one of the plurality of constraints 516 recesses, the force applied by compliant fourth link 514 causes the relative arrangement of at least some of links 508, 510, 512, 514 to adjust such that the one constraint proximate to the recess maintains engagement with head 10. Further, in one example, compliant fourth link 514 and the joints formed by couplers 520 and 522 cause the relative arrangement of at least some of links 508, 510, 512, 514 to adjust such that the one constraint proximate to the recess maintains engagement with head 10 and that all constraints exert a generally uniform force against head 10. In a preferred example, compliant fourth link 514 and the joints formed by couplers 520 and 522 cause the relative arrangement of at least some of links 508, 510, 512, 514 to adjust such that the one constraint proximate to the recess maintains engagement with head 10, each constraint 516, 518 exerts a generally uniform force against head 10, and each constraint 516, 518 continues to exert generally the same amount of force against head 10 as prior to the recess of head 10 occurring.

Head restraint member 506 when applied to head 10 should exert sufficient force to limit the movement of head 10 relative to head restraint member 506. In one example a force of at least about 30 pounds should be exerted by head restraint member 506. In another example, a force of at least about 40 pounds should be exerted by head restraint member 506. In a further example, a force of at least about 50 pounds should be exerted by head restraint member 506. In yet a further example, a force of at least about 60 pounds should be exerted by head restraint member 506. In yet another example, a force of between about 30 pounds to about 100 pounds should be executed by head restraint member 506. In still another example, a force of between about 40 pounds to about 100 pounds should be executed by head restraint member 506. In yet still another example, a force of between about 50 pounds to about 100 pounds should be executed by head restraint member 506. In still a further example, a force of between about 50 pounds to about 60 pounds should be executed by head restraint member 506.

Referring to FIG. 14, body restraint member of 505 is secured or engaged to body 12 of the person. In a preferred example, body restraint member 505 is secured to the torso of the person. Body restraint member 505 and head restraint member 506 are coupled together to limit the movement of head 10 relative to body or torso 12. In the illustrated embodiment, a superstructure 508 of body restraint member 505 is coupled to third link 512 of link system 504 of head restraint member 506. In one example, the each of the exemplary superstructures described above in connection with restraint system or apparatus 100 may be coupled to head restraint member 506 to couple head 10 to torso 12.

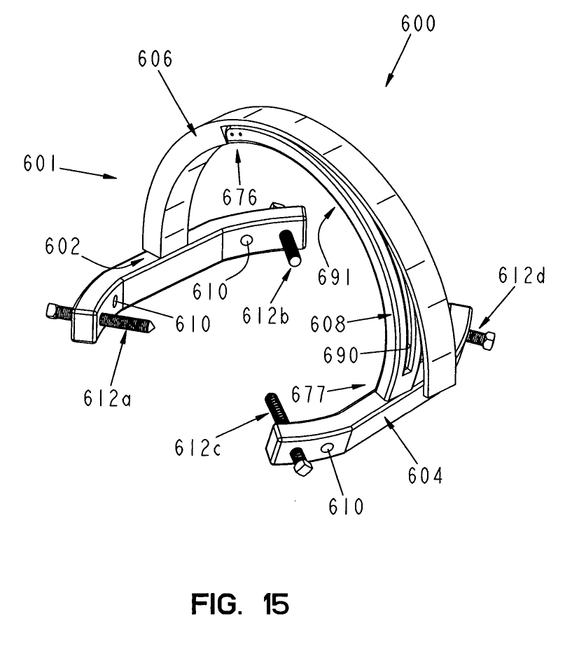

Referring to FIGS. 15-18, an exemplary head restraint system 600 according to the present invention is shown. Head restraint system 600 includes a link system 601 and constraints 612. Link system 601 includes a first link 602, a second link 604, a third link 606, and a fourth link 608. Head restraint member 600 has the same kinematic structure 502 as the head restraint member 506 shown in FIG. 14.

Referring to FIG. 15, first link 602 and second link 604 each include a plurality of apertures 610 each configured to receive plurality of constraints or pins 612. As illustrated in FIG. 15, first link 602, in one example, includes two constraints 612a and 612b. It is contemplated that constraints 612a, 612b may be received in other apertures 610 and/or that additional constraints 612 are received in additional apertures 610. Second link 604 includes plurality of apertures 610 each configured to receive constraint 612. As illustrated in FIG. 15, second link 604, in one example, includes two constraints 612c and 612d. It is contemplated that constraints 612c, 612d may be received in other apertures 610 and/or that additional constraints 612 are received in additional apertures 610.

Preferably, constraints 612a, 612b, 612c, 612d are pins which are rigidly secured to the respective link of first link 602 and second link 604 such that the pins or constraints 612a, 612b, 612c, 612d do not move relative to the respective link of first link 602 and second link 604 once head restraint member 600 is applied to head 10. In one example, constraints 612a, 612b, 612c, 612d are permanently rigidly coupled to link system 601 of head restraint member 600 by welding, gluing, epoxying, mechanical fastening, or other suitable means for permanently rigidly coupling constraints 612a, 612b, 612c, 612d to head restraint member 600. In one variation, constraints 612a, 612b, 612c, 612d and the respective first link 602 and second link 604 are integrally formed or made as a single component.

In another example, each pin or constraint 612a, 612b, 612c, 612d is moveably coupled to the respective link of first link 602 and second link 604 during the application of head restraint member 600 to head 10 and rigidly coupled to the respective link of first link 602 and second link 604 such that the respective pin or constraint 612a, 612b, 612c, 612d does not move relative to the respective link of first link 602 and second link 604 once head restraint member 600 is applied to head 10. For example, pins 612a, 612b, 612c, 612d may be threadably received in respective apertures 610 of first link 602 and second link 604 such that pins 612a, 612b, 612c, 612d are moveable relative to link system 601. Pins 612a, 612b, 612c, 612d are then rigidly coupled to link system 601 with a lock nut.

Both of first link 602 and second link 604 include a faceted longitudinal extent approximating an arcuate longitudinal extent which in turn is an approximation of the curvature of the left half of the head and the right half of the head, respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 602 and second link 604 and/or orientation of apertures 610 is such that pins or constraints 612a, 612b, 612c, 612d are generally normal to head 10 when head restraint system 600 is applied to head 10.

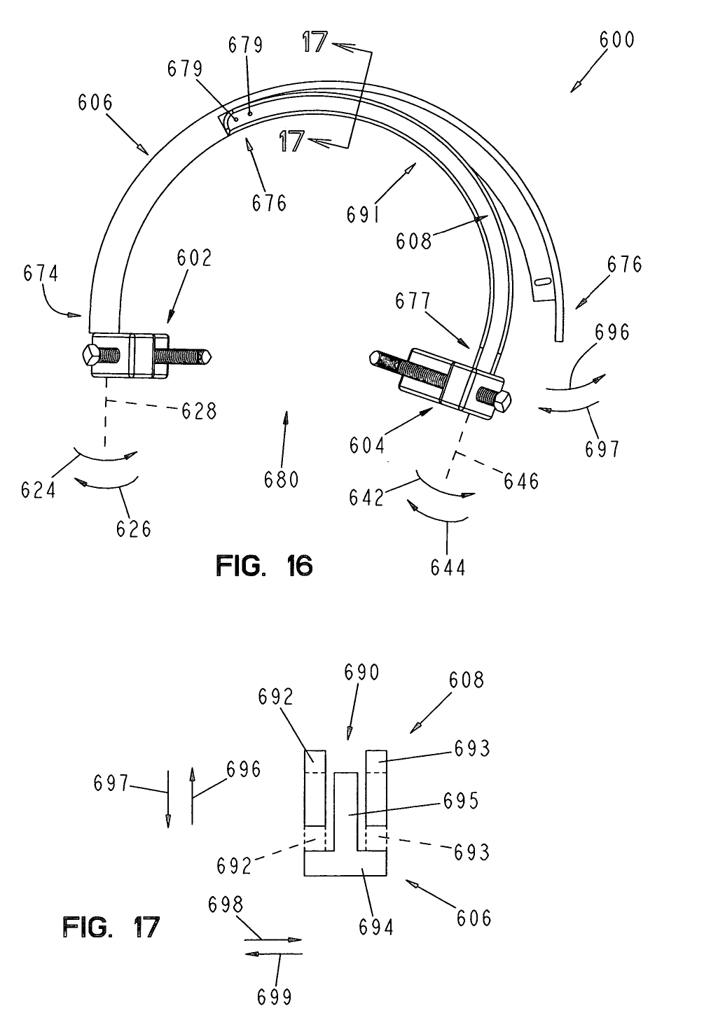

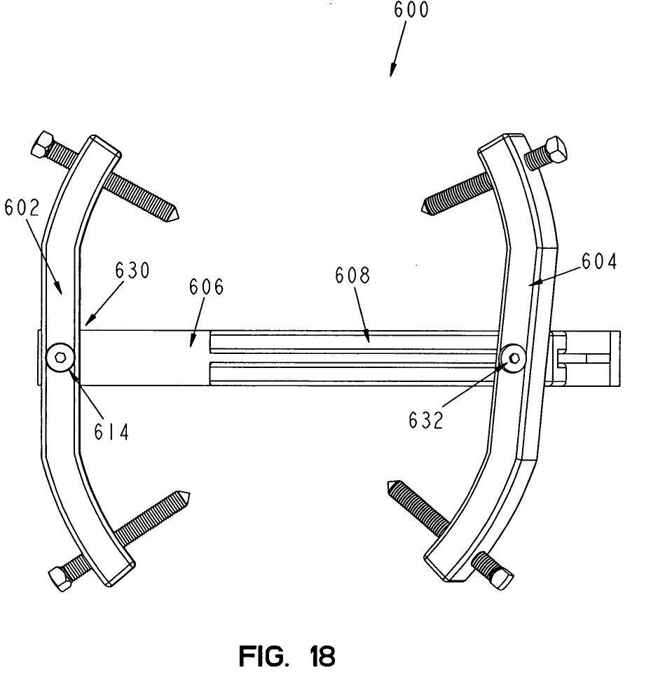

First link 602 is further coupled to third link 606 by a coupler or pin 614 (see FIG. 18). Pin 614 includes a shoulder portion and a threaded portion. First link 602 includes an aperture sized to receive the shoulder portion of coupler 614. Third link 606 includes a threaded aperture which is sized to threadably receive the threaded portion of coupler 614. Referring to FIG. 16, once first link 602 is coupled to third link 606 by coupler 614, first link 602 is constrained to move in a single degree of freedom, namely to rotate in directions 624 and 626 about an axis 628 of coupler 614. As such, coupler 614 creates a revolute joint 630 (see FIG. 18) between first link 602 and third link 606. In alternative examples, the joint between the first link and the third link is one of a prismatic joint or a slider joint.

Second link 604 is further coupled to fourth link 608 by a coupler or pin 632 (see FIG. 18). Pin 632 includes a shoulder portion and a threaded portion. Second link 604 includes an aperture sized to receive the shoulder portion of coupler 632. Fourth link 608 includes a threaded aperture which is sized to threadably receive the threaded portion of coupler 632. Referring to FIG. 16, once second link 604 is coupled to fourth link 608 by coupler 632, second link 604 is constrained to move in a single degree of freedom, namely to rotate in directions 642 and 644 about an axis 646 of coupler 632. As such, coupler 632 creates a revolute joint 648 between second link 604 and fourth link 608. In alternative examples, the joint between the second link and the fourth link is one of a prismatic joint or a slider joint.

Compliant fourth link 608 is further coupled to third link 606. Compliant fourth link 608 preferably is rigidly coupled to third link 606 and is further made of a compliant material such that compliant fourth link 608 either directly or through third link 606 exerts a force on links 602, 604 which in turn causes constraints 612a, 612b, 612c, 612d to remain in contact with head 10.

In one example, complaint fourth link 608, is made from a non-magnetic material such that complaint link 608 does not interfere with MRI imaging of head 10. One example non-magnetic material is a glass-filled epoxy available from Composiflex located at 8100 Hawthorne Drive Erie Pa. 16509. In another example, compliant fourth link 608 is made from any material which is capable of exerting a force resulting in constraints 612a, 612b, 612c, 612d engaging head 10 over time.

Compliant fourth link 608 has a relaxed state and an unrelaxed state. Compliant fourth link 608, in a preferred embodiment, is not in a relaxed state when head restraint member 600 is applied to head 10. Compliant fourth link 608 is rigidly coupled to third link 606 at a first end 676 and a second end 677 of compliant link 608 is free to move relative to third link 606. Second end 677 of compliant fourth link 608 is biased such that second link 604 is moved toward first link 602.

Compliant fourth link 608, in the example shown in FIGS. 15-18, is generally U-shaped and includes a channel 690 (see FIG. 17) along a longitudinal extent. Channel 690 does not extend along the entire length of compliant fourth link 608. On the contrary, channel 690 extends from first end 676 along a central portion 691 (see FIG. 15) of fourth link 608 and is capped by second end 677. Referring to FIG. 17, along central portion 691 which includes channel 690, compliant fourth link 608 includes a first portion 692 and a second portion 693 separated by channel 690. First portion 692 and second portion 693 of fourth link 608 are rigidly coupled to third link 606 at first end 676 by fasteners 679 (see FIG. 16).

Referring to FIG. 17, third link 606 in the same region as central portion 691 of fourth link 608 is T-shaped and includes a first portion 694 and a second portion 695. Second portion 695 of third link 606 is sized to be received within channel 690 of fourth link 608. Second portion 695 permits second end of fourth link 608 to move generally in directions 696, 697 and limits the movement of second end 677 of fourth link 608 in directions 698, 699.

As shown in FIGS. 16 and 17, fourth link 608 in a relaxed state. However, when fourth link 608 is in an unrelaxed state second portion 695 of third link 606 is further received by channel 690. Referring to FIG. 17, fourth link 608 is shown in an exemplary unrelaxed state in phantom.

The compliance of fourth link 608 in combination with the joints formed by couplers 630 and 648 permit link system 601 to adapt to small changes in the geometry of the head. For instance, if the portion of head 10 engaged by one of the plurality of constraints 612a, 612b, 612c, 612d recesses, the force applied by compliant fourth link 608 causes the relative arrangement of at least some of links 612a, 612b, 612c, 612d to adjust such that the one constraint proximate to the recess maintains engagement with head 10. Further, in one example, compliant fourth link 608 and the joints formed by couplers 630 and 648 cause the relative arrangement of at least some of links 602, 604, 606, 608 to adjust such that the one constraint proximate to the recess maintains engagement with head 10 and that all constraints 612a, 612b, 612c, 612d exert a generally uniform force against head 10. In a preferred example, compliant fourth link 608 and the joints formed by couplers 630 and 648 causes the relative arrangement of at least some of links 602, 604, 606, 608 to adjust such that the one constraint proximate to the recess maintains engagement with head 10, each constraint 612a, 612b, 612c, 612d exerts a generally uniform force against head 10, and each constraint 612a, 612b, 612c, 612d continues to exert generally the same amount of force against head 10 as prior to the recess of head 10 occurring.

Referring to FIG. 16, third link 606 spans an opening 680 of head restraint member 600 sized to receive a head of the person. It is contemplated that third link 606 further includes a first mounting bracket, similar to first mounting bracket 482, located at first end 674 and a second mounting bracket, similar to second mounting bracket 484, located at a second end 676. The mounting brackets, respectively, are configured to couple third link 606 to a body restraint device, such as body restraint member 205 of FIG. 4. As such, third link 606 alone provides the coupling between head restraint member 600 and body restraint member 205. Therefore, the movement of first link 602 relative to third link 606, second link 604 relative to compliant fourth link 608, and compliant fourth link 608 relative to third link 606 is isolated from the coupling of head restraint member 600 to body restraint member 205.

It is contemplated that the placement of constraints 612a, 612b, 612c, 612d may be balanced or unbalanced. In one example, constraints 612a and 612b are generally equidistant from joint 630 and constraints 612c and 612d are generally equidistance from joint 648. In one variation, the distance from joint 630 to each of constraints 612a and 612b is generally equal to the distance from joint 648 to each of constraints 612c and 612d. In another variation, the distance from joint 630 to each of constraints 612a and 612b is generally not equal to the distance from joint 648 to each of constraints 612c and 612d. In another example, at least one pair of constraints 612a and 612b and constraints 612c and 612d are generally non-equidistant from the respective joint 630 and joint 648. In one variation, the distance from joint 630 to each of constraints 612a and 612b is generally equal and the distance from joint 648 to each of constraints 612c and 612d is generally not equal. In another variation, the distance from joint 630 to each of constraints 612a and 612b is generally not equal and the distance from joint 648 to each of constraints 612c and 612d is generally not equal.

Head restraint system 600 is applied to a head of the person as follows. In a first exemplary method, pins 612a, 612b, 612c, 612d are assembled to respective links 602, 604 such that pins 612a, 612b, 612c, 612d extend inward from links 602 and 604 by a predetermined amount. Complaint fourth link 608 is moved generally in direction 697 such that the spacing of opening 680 between first link 602 and second link 604 is increased. The movement of fourth link 608 in direction 697 further causes compliant fourth link 608 to be further stretched. In an example a spacer tool is used to stretch compliant fourth link 608 and to maintain the orientation of compliant fourth link 608 relative to third link 606 prior to applying head restraint member 600 to head 10.

Head restraint member 600 is placed over head 10. Compliant fourth link 608 is gradually allowed to move in direction 696 such that pins 612a, 612b, 612c, 612d are brought into engagement with head 10. At this point, head restraint member 600 is applied to head 10. The applied head restraint member 600 is further assembled to a body restraint device, such as body restraint member 205 to limit the movement of head 10 relative to the torso 12 of the person.

It should be noted that the force exerted by pins 612a, 612b, 612c, 612d is governed by the force exerted by compliant fourth link 608. In the illustrated example, the force exerted by pins 612a, 612b, 612c, 612d is governed by the characteristics of the elastic material of fourth link 608. As such, the orthopedic surgeon may adjust the force exerted by pins 612a, 612b, 612c, 612d by utilizing a compliant fourth link 608 having certain predetermined characteristics. Alternatively, in embodiments where pins 612a, 612b, 612c, 612d are threadably coupled to first link 602 and second link 604, the force exerted by the respective pins 612a, 612b, 612c, 612d may be adjusted by either advancing the respective pin in the respective aperture 610 to increase the force or by retracting the respective pin outward of respective aperture 610 to decrease the force. It should be noted that the advancement or retraction of a single constraint 612a, 612b, 612c, 612d results in a corresponding increase or decrease in force for all of constraints 612a, 612b, 612c, 612d due to the arrangement of links 602, 604, 606, and compliant fourth link 608.

Head restraint member 600 when applied to head 10 should exert sufficient force to limit the movement of head 10 relative to head restraint member 600. In one example a force of at least about 30 pounds should be exerted by head restraint member 600. In another example, a force of at least about 40 pounds should be exerted by head restraint member 600. In a further example, a force of at least about 50 pounds should be exerted by head restraint member 600. In yet a further example, a force of at least about 60 pounds should be exerted by head restraint member 600. In yet another example, a force of between about 30 pounds to about 100 pounds should be executed by head restraint member 600. In still another example, a force of between about 40 pounds to about 100 pounds should be executed by head restraint member 600. In yet still another example, a force of between about 50 pounds to about 100 pounds should be executed by head restraint member 600. In still a further example, a force of between about 50 pounds to about 60 pounds should be executed by head restraint member 600.

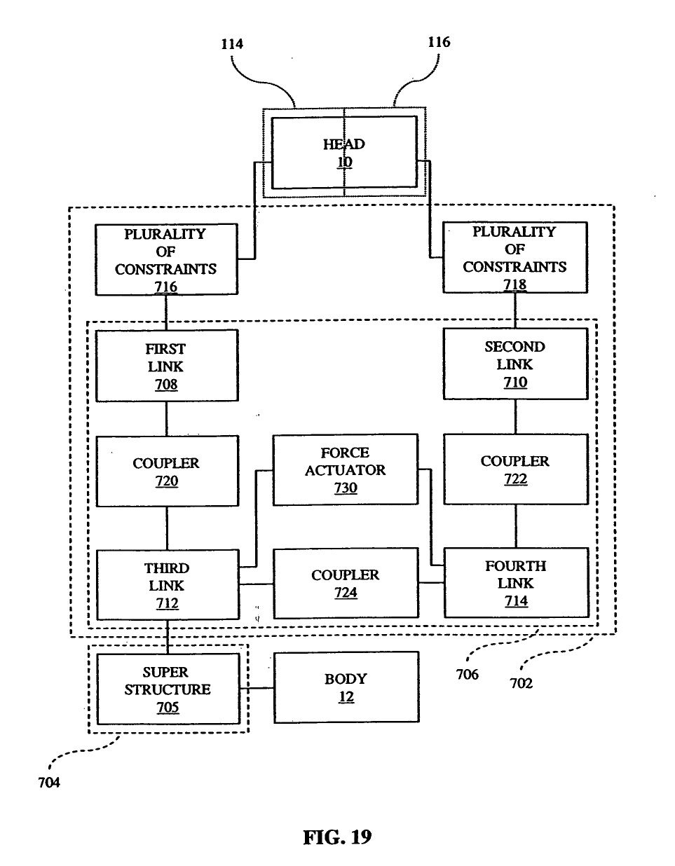

Referring to FIG. 19, an exemplary restraint system or apparatus 700 according to the present invention is shown. Restraint system 700 includes a head restraint member 702 for engaging a head or skull 10 of a person and a body restraint member 704 for securing to the body 12 of the person. In a preferred example, body restraint member 704 is secured to the torso of the person. Head restraint member 702 includes a link system 706, a first plurality of constraints 716, and a second plurality of constraints. An exemplary kinematic structure 701 for head restraint member 702 of restraint system 700 is shown in FIG. 19. Link system 706 includes a first link 708, a second link 710, a third link 712 and a four link 714. Head restraint member 702 and body restraint member 704 are coupled together to limit the movement of head 10 relative to body or torso 12. In the illustrated embodiment, third link 712 of link system 706 is rigidly coupled to a superstructure 705 of body restraint member 704.

Link system 706 is coupled to a first plurality of constraints 716 and a second plurality of constraints 718. First plurality of constraints 716 are located such that the first plurality of constraints 716 engage head 10 generally along a first half 114 of head 10. Second plurality of constraints 718 are located such that the second plurality of constraints 718 engage head 10 generally along a second half 116 of head 10.

Link system 706 exerts a force on head 10 of the person through first plurality of constraints 716 and second plurality of constraints 718 such that head 10 is generally fixed relative to head restraint member 702. It should be understood that if head 10 is fixed relative to head restraint member 702, then head 10 is fixed relative to torso 12 due to body restraint member 704 being secured to torso 12 and body restraint member 704 being rigidly coupled to head restraint member 702.

In a preferred embodiment of head restraint member 702, the first plurality of constraints 716 and the second plurality of constraints 718 are comprised of pins. Each pin includes a pin tip configured to pierce the skin on the head of the person and embed into the skull of the person. Exemplary pins include pins sold in combination with the Generation 80 cervical product available from Jerome Medical located at 305 Harper Drive, Moorestown, N.J. 08057-3239, pins and pin mounts disclosed in US Published Application No. 20020151831A1 to Stamper et al., filed Feb. 2, 2001, the disclosure of which is incorporated by reference herein, and the pins disclosed in paper titled “Structural Behavior of the Halo Orthosis Pin-Bone Interface: Biomechanical Evaluation of Standard and Newly Designed Stainless Steel Halo Fixation Pins,” by Garfin, S.,et al., published in Spine, Vol. 11, No. 10, 1986, the disclosure of which is incorporated by reference herein.

First link 708 and second link 710 are each capable of supporting a plurality of constraints 716, 718, respectively. Preferably, constraints 716, 718 are pins which are rigidly secured to the respective link of first link 708 and second link 710 such that the pins or constraints 716, 718 do not move relative to link system 706 during the time head restraint member 702 is applied to head 10. In one example, constraints 716, 718 are permanently rigidly coupled to link system 706 of head restraint member 702 by welding, gluing, epoxying, mechanical fastening, or other suitable means for permanently rigidly coupling constraints 716, 718 to head restraint member 702. In one variation, constraints 716, 718 and the respective portions of link system 706 are integrally formed or made as a single component. In another example, each pin or constraint 716, 718 is moveably coupled to the link system 706 during the assembling of head restraint member 702 to head 10 and rigidly coupled to the link system 706 such that pin or constraint 716, 718 does not move relative to link system 706 once head restraint member 702 is assembled to head 10.

First link 708 is further coupled to third link 712 through a coupler 720 forming a joint between first link 708 and third link 712. Second link 710 is further coupled to fourth link 714 through a coupler 722 forming a joint between second link 710 and fourth link 714. Preferably, both of the joints formed by couplers 720 and 722 permit generally only a single degree of motion between the respective links of the joint. As such, first link 708 is capable of moving in only one degree of freedom relative to third link 712 and second link 710 is capable of moving in only one degree of freedom relative to fourth link 714. In one example, the joints formed by couplers 720 and 722 are revolute joints. In alternative examples, the joints are one of prismatic joints, or slider joints.

Third link 712 is further coupled to fourth link 714 through a coupler 724 forming a joint between third link 712 and fourth link 714. Preferably, the joint formed by coupler 724 permits generally only a single degree of motion between the respective links 712, 714. As such, fourth link 714 is capable of moving in only one degree of freedom relative to third link 712. In one example, the joint formed by coupler 724 is a revolute joint. In alternative examples, the joint is one of a prismatic joint or a slider joint.

Head restraint member 702 further includes a force actuator 730. Force actuator 730 is coupled to third link 712 and to fourth link 714. In one example, force actuator 730 exerts a tension force to links 712, 714. In another example, force actuator 730 exerts a compressive force to links 712, 714.

In one embodiment, force actuator 730 is coupled to each of third link 712 and fourth link 714 such that force actuator 730 has one degree of freedom relative to each of the third link 712 and fourth link 714. In one example, force actuator 730 is translatably coupled to each of third link 712 and fourth link 714. In another embodiment, force actuator 730 is rotatably coupled to each of third link 712 and fourth link 714. In yet another embodiment, force actuator 730 is translatably coupled to one of third link 712 and fourth link 714 and rotatably coupled to the other of third link 712 and fourth link 714. In still a further embodiment, force actuator 730 is at least one of rotatably coupled and/or translatably coupled to one of third link 712 and fourth link 714 and is not coupled to the other of third link 712 and fourth link 714.

In one embodiment, force actuator 730 preferably orients fourth link 714 relative to third link 712. In one example, fourth link 714 is rotatably coupled to third link 712. Force actuator 730 controls the orientation of fourth link 714 relative to third link 712, such that fourth link 714 is not moveably relative to third link 712 without a change in the force exerted by force actuator 730. By increasing the force exerted by force actuator 730 the force exerted by constraints 716, 718 against head 10 may be increased. By decreasing the force exerted by force actuator 730 the force exerted by constraints 716, 718 against head 10 may be decreased. Further, due to the characteristics of the joints formed by couplers 720, 722, 724 (only a single degree of freedom) the force exerted by each constraint 716, 718 is generally uniform compared to the remaining constraints 716, 718 and any increase or decrease in force applied by constraints 716, 718 as the result of a change in the amount of tension applied by force actuator 730 is generally equal for each constraint 716, 718 compared to the remaining constraints 716, 718. As such, force actuator 730 provides for the simultaneous increasing or decreasing of force exerted by constraint 716, 718 by adjusting force actuator 730.

Head restraint member 702 when applied to head 10 should exert sufficient force to limit the movement of head 10 relative to head restraint member 702. In one example a force of at least about 30 pounds should be exerted by head restraint member 702. In another example, a force of at least about 40 pounds should be exerted by head restraint member 702. In a further example, a force of at least about 50 pounds should be exerted by head restraint member 702. In yet a further example, a force of at least about 60 pounds should be exerted by head restraint member 702. In yet another example, a force of between about 30 pounds to about 100 pounds should be executed by head restraint member 702. In still another example, a force of between about 40 pounds to about 100 pounds should be executed by head restraint member 702. In yet still another example, a force of between about 50 pounds to about 100 pounds should be executed by head restraint member 702. In still a further example, a force of between about 50 pounds to about 60 pounds should be executed by head restraint member 702.

Referring to FIG. 19, body restraint member of 704 is secured or engaged to body 12 of the person. In a preferred example, body restraint member 704 is secured to the torso of the person. Body restraint member 704 and head restraint member 702 are coupled together to limit the movement of head 10 relative to body or torso 12. In the illustrated embodiment, a superstructure 705 of body restraint member 704 is coupled to third link 712 of link system 706 of head restraint member 702. In one example, each of the exemplary superstructures described above in connection with restraint system or apparatus 100 may be coupled to head restraint member 702 to couple head 10 to torso 12.

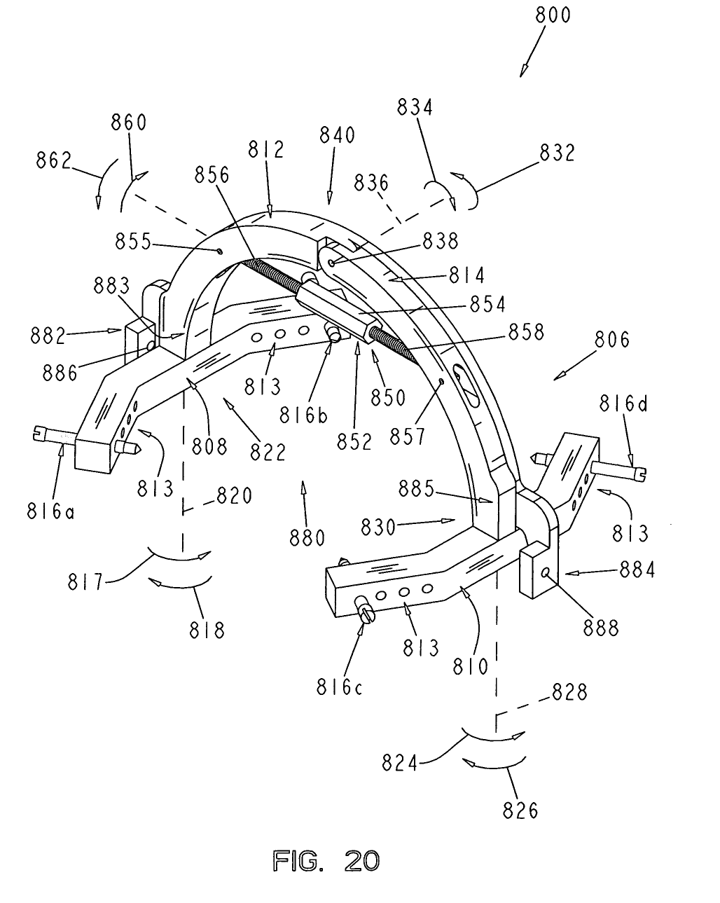

Referring to FIG. 20, an exemplary head restraint system 800 according to the present invention is shown having the kinematic structure 701 of the head restraint member 702 shown in FIG. 19. Head restraint system 800 includes a link system 806 and constraints 816. Link system 806 includes a first link 808, a second link 810, a third link 812, and a fourth link 814.

First link 808 and second link 810 each include a plurality of apertures 813 to receive plurality of constraints or pins 816. As illustrated in FIG. 20, first link 808, in one example, includes two constraints 816a and 816b. It is contemplated that constraints 816a, 816b may be received in other apertures 813 and/or that additional constraints 816 are received in additional apertures 813. Second link 810, in one example, includes two constraints 816c, 816d. It is contemplated that constraints 816c, 816d may be received in other apertures 813 and/or that additional constraints 816 are received in additional apertures 813.

Preferably, constraints 816a, 816b, 816c, 816d are pins which are rigidly secured to the respective link of first link 808 and second link 810 such that the pins or constraints 816a, 816b, 816c, 816d do not move relative to the respective link of first link 808 and second link 810 once head restraint member 800 is assembled to head 10. In one example, constraints 816a, 816b, 816c, 816d are permanently rigidly coupled to link system 806 of head restraint member 800 by welding, gluing, epoxying, mechanical fastening, or other suitable means for permanently rigidly coupling constraints 816a, 816b, 816c, 816d to head restraint member 800. In one variation, constraints 816a, 816b, 816c, 816d and the respective first link 808 and second link 810 are integrally formed or made as a single component.

In another example, each pin or constraint 816a, 816b, 816c, 816d is moveably coupled to the respective link of first link 808 and second link 810 during the application of head restraint member 800 to head 10 and rigidly coupled to the respective link of first link 808 and second link 810 such that the respective pin or constraint 816a, 816b, 816c, 816d does not move relative to the respective link of first link 808 and second link 810 once head restraint member 800 is assembled to head 10. For example, pins 816a, 816b, 816c, 816d may be threadably received in respective apertures of first link 808 and second link 810 such that pins 816a, 816b, 816c, 816d are moveable relative to link system 806. Pins 816a, 816b, 816c, 816d are then rigidly coupled to link system 806 with a lock nut.

Both of first link 808 and second link 810 include a faceted longitudinal extent approximating an arcuate longitudinal extent that in turn is an approximation of the curvature of the left half of the head and the right half of the head, respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 808 and second link 810 and/or orientation of apertures 813 is such that pins or constraints 816a, 816b, 816c, 816d are generally normal to head 10.

First link 808 is further coupled to third link 812 by a coupler or pin. Once first link 808 is coupled to third link 812 by the coupler, first link 808 is constrained to move in a single degree of freedom, namely to rotate in directions 817 and 818 about an axis 820 of the coupler. As such, the coupler creates a revolute joint 822 between first link 808 and third link 812. In alternative examples, the joint between the first link and the third link is one of a prismatic joint or a slider joint.

Second link 810 is further coupled to fourth link 814 by a coupler or pin. Once second link 810 is coupled to fourth link 814 by the coupler, second link 810 is constrained to move in a single degree of freedom, namely to rotate in directions 824 and 826 about an axis 828 of the coupler. As such, the coupler creates a revolute joint 830 between second link 810 and fourth link 814. In alternative examples, the joint between the second link and the fourth link is one of a prismatic joint or a slider joint.

Third link 812 and fourth link 814 are coupled together such that fourth link 814 is constrained to move in a single degree of freedom, namely to rotate in directions 832 and 834 about an axis 836 of a coupler 838 which couples third link 812 and fourth link 814. Once fourth link 814 is coupled to third link 812 by coupler 838, fourth link 814 is constrained to rotate in directions 832 and 834. As such, coupler 838 creates a revolute joint 840 between third link 812 and fourth link 814. In alternative examples, joint 840 between the third link and the fourth link is one of a prismatic joint or a slider joint.

Head restraint member 800 further includes a force actuator 850. In one embodiment, force actuator or force applier 850 applies or exerts at least one of a tension force or a compressive force on links 1006, 1008. Force actuator 850 is coupled to third link 812 and to fourth link 814. Force actuator 850 is coupled to each of third link 812 and fourth link 814 such that force actuator 850 has one degree of freedom relative to each of the third link 812 and fourth link 814. As shown, force actuator 850 is coupled to each of third link 812 and fourth link 814 such that force actuator 850 is able to rotate relative to third link 812 and fourth link 814. In an alternative embodiment, force actuator 850 is translatably coupled to each of third link 812 and fourth link 814. In yet another alternative embodiment, force actuator 850 is translatably coupled to one of third link 812 and fourth link 814 and rotatably coupled to the other third link 812 and fourth link 814. In a further alternative embodiment, force actuator 850 has at least two degrees of freedom relative to at least one of third link 812 and fourth link 814. In still a further embodiment, force actuator 850 is at least one of rotatably coupled and/or translatably coupled to one of third link 812 and fourth link 814 and is not coupled to the other of third link 812 and fourth link 814.

Force actuator 850, in the illustrated embodiment, is a turnbuckle 852. Turnbuckle 852 applies a tension force on links 812, 814. Turnbuckle 852 includes a central component 854 which is internally threaded and a pair of threaded rods 856, 858. Rods 856, 858 are rotatably coupled to third link 812 and fourth link 814 by pins 855, 857, respectively. Rods 856, 858 are further threadably received by central component 854. By rotating central component 854 in one of directions 860, 862, rods 856, 858 are further advanced within central component 854. The advancement of rods 856, 858 within central component 854 results in the separation of pins 855, 857 being reduced and in turn fourth link 814 is rotated in direction 834 such that second link 810 moves towards first link 808. By rotating central component 854 in the other direction of directions 860, 862, rods 856, 858 are further retracted from within central component 854. The retraction of rods 856, 858 from within central component 854 results in the separation of pins 855, 857 being increased and in turn fourth link 814 is rotated in direction 832 such that second link 810 moves away from first link 808.

As stated above, force actuator 850 preferably orients fourth link 814 relative to third link 812. Force actuator 850 controls the orientation of fourth link 814 relative to third link 812 about the joint formed by coupler 838, such that fourth link 814 is not moveably relative to third link 812 without a change in the tension exerted by force actuator 850. By increasing the tension exerted by force actuator 850 the force exerted by constraints 816a, 816b, 816c, 816d against head 10 may be increased. By decreasing the tension exerted by force actuator 850 the force exerted by constraints 816a, 816b, 816c, 816d against head 10 may be decreased. Further, due to the characteristics of the joints formed by couplers 822, 830, 838 (only a single degree of freedom) the force exerted by each constraint 816a, 816b, 816c, 816d is generally uniform compared to the remaining constraints 816a, 816b, 816c, 816d and any increase or decrease in force applied by constraints 816a, 816b, 816c, 816d as the result of a change in the amount of tension applied by force actuator 850 is generally equal for each constraint 816a, 816b, 816c, 816d compared to the remaining constraints 816a, 816b, 816c, 816d. As such, force actuator 850 provides for the simultaneous increasing or decreasing of force exerted by constraint 816a, 816b, 816c, 816d by adjusting force actuator 850.

Third link 812 spans an opening 880 of head restraint member 800 sized to receive the head of the person. Third link 812 further includes a first mounting bracket 882 located at first end 883 of third link 812 and a second mounting bracket 884 located at a second end 885 of third link 812. Mounting brackets 882, 884 respectively include mounting apertures 886 and 888. Mounting apertures 886 and 888 are configured to receive fasteners to couple third link 812 to a body restraint device, such as body restraint member 205 of FIG. 4. As such, third link 812 alone provides the coupling between head restraint member 800 and body restraint member 205. Therefore, the movement of first link 808 relative to third link 812, second link 810 relative to fourth link 814, and fourth link 814 relative to third link 812 is isolated from the coupling of head restraint member 800 to body restraint member 205.

It is contemplated that the placement of constraints 816a, 816b, 816c, 816d may be balanced or unbalanced. In one example, constraints 816a and 816b are generally equidistant from joint 822 and constraints 816c and 816d are generally equidistance from joint 830. In one variation, the distance from joint 822 to each of constraints 816a and 816b is generally equal to the distance from joint 430 to each of constraints 816c and 816d. In another variation, the distance from joint 822 to each of constraints 816a and 816b is generally not equal to the distance from joint 830 to each of constraints 816c and 816d. In another example, at least one pair of constraints 816a and 816b and constraints 816c and 816d are generally non-equidistant from the respective joint 822 and joint 830. In one variation, the distance from joint 822 to each of constraints 816a and 816b is generally equal and the distance from joint 830 to each of constraints 816c and 816d is generally not equal. In another variation, the distance from joint 822 to each of constraints 816a and 816b is generally not equal and the distance from joint 830 to each of constraints 816c and 816d is generally not equal.

Head restraint system 800 is applied to a head of the person as follows. In a first exemplary method, pins 816a, 816b, 816c, 816d are assembled to respective links 808, 810 such that pins 816a, 816b, 816c, 816d extend inward from links 808 and 810 by a predetermined amount. Head restraint member 800 is placed over head 10. Fourth link 814 is gradually rotated in direction 834 such that pins 816a, 816b, 816c, 816d are brought into engagement with head 10. Fourth link 814 is rotated in direction 834 by shortening the length of force actuator 850. The shortening of turnbuckle 852 results in all four constraints 816a, 816b, 816c, 816d being simultaneously engaged with the head. At this point, head restraint member 800 is applied to head 10. The applied head restraint member 800 is further assembled to a body restraint device, such as body restraint member 205 to limit the movement of head 10 relative to the torso 12 of the person.

It should be noted that the force exerted by pins 816a, 816b, 816c, 816d is governed by the force exerted by force actuator 850 on third link 812 and fourth link 814. In the illustrated example, the force exerted by pins 816a, 816b, 816c, 816d is governed by the length of turnbuckle 852. As such, the orthopedic surgeon may adjust the force exerted by all pins 816a, 816b, 816c, 816d by adjusting the length of turnbuckle 852.

Head restraint member 800 when applied to head 10 should exert sufficient force to limit the movement of head 10 relative to head restraint member 800. In one example a force of at least about 30 pounds should be exerted by head restraint member 800. In another example, a force of at least about 40 pounds should be exerted by head restraint member 800. In a further example, a force of at least about 50 pounds should be exerted by head restraint member 800. In yet a further example, a force of at least about 60 pounds should be exerted by head restraint member 800. In yet another example, a force of between about 30 pounds to about 100 pounds should be executed by head restraint member 800. In still another example, a force of between about 40 pounds to about 100 pounds should be executed by head restraint member 800. In yet still another example, a force of between about 50 pounds to about 100 pounds should be executed by head restraint member 800. In still a further example, a force of between about 50 pounds to about 60 pounds should be executed by head restraint member 800.

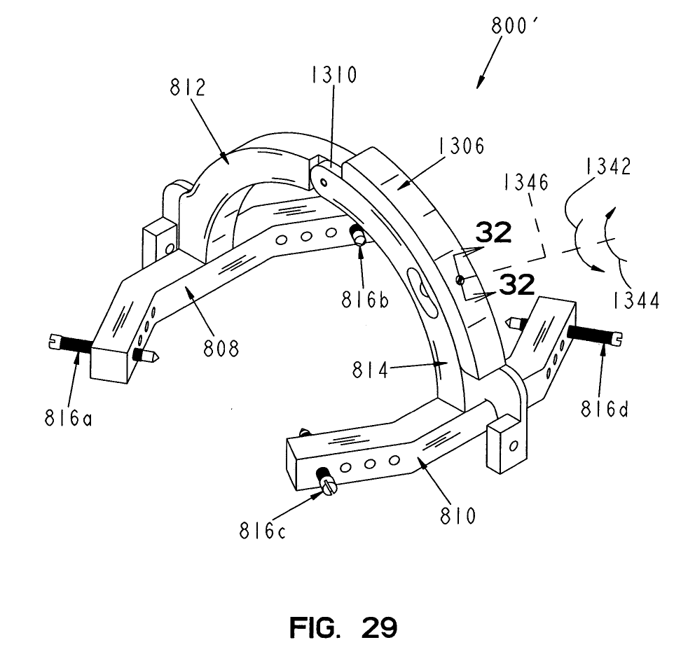

Referring to FIGS. 29-32, a head restraint system 800′ is shown. Head restraint system 800′ is generally similar to head restraint system 800 except that force actuator 850 of head restraint system 800 is replaced by a force actuator 850′. Further, third link 812 and fourth link 814 are modified to accommodate force actuator 850′. Force actuator or force applier 850 exerts a tension force on links 812, 814 while force actuator or force applier 850′ exerts a compressive force on links 812, 814.

Referring to FIG. 32, force actuator 850′ includes a first member 1300 and a second member 1302. By adjusting an overall length 1304 of first member 1300 and second member 1302 the amount of force exerted by force actuator 850′ on links 812, 814 may be increased or decreased. By shortening length 1304, the force exerted by force actuator 850′ may be decreased. By enlarging length 1304, the force exerted by force actuator 850′ may be increased.

Referring to FIG. 29, third link 812 further includes an upper portion 1306 sized such that a lower surface 1308 (see FIG. 32) of upper portion 1306 overlays an upper surface 1310 of fourth link 814. Upper portion 1306 includes an aperture 1312 sized to receive second member 1302. In the illustrated embodiment, second portion 1302 is a screw including a tool engaging portion 1314, a shoulder portion 1316, and a threaded portion 1318. Aperture 1312 includes a first portion 1320 sized to receive tool engaging portion 1314 of screw 1302 and a second larger diameter portion 1322 sized to receive shoulder portion 1316 and a beveled washer 1324.

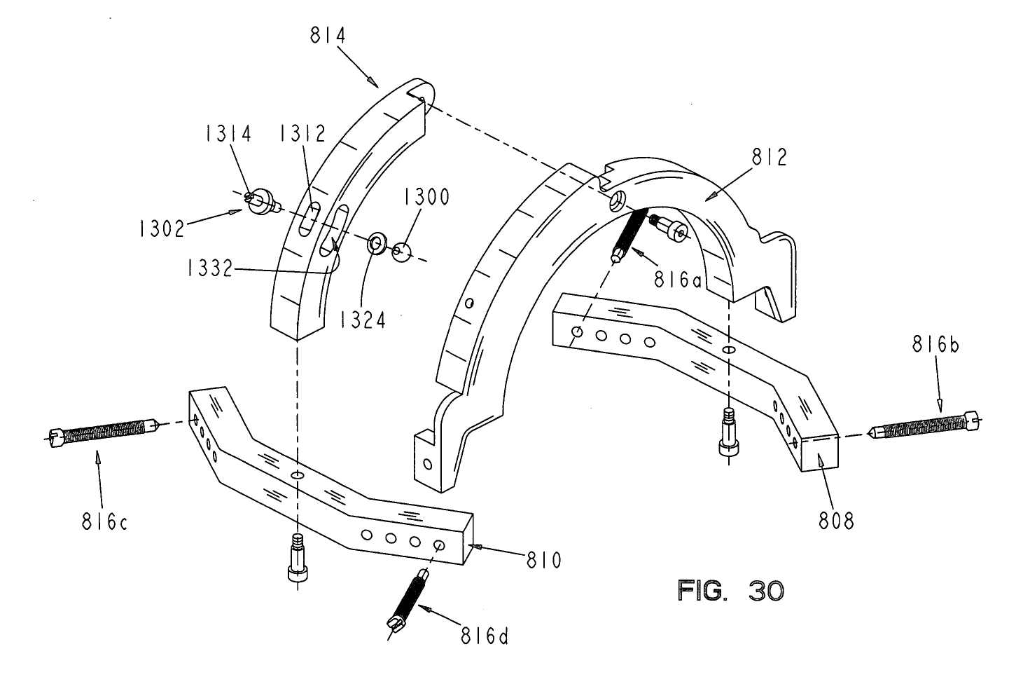

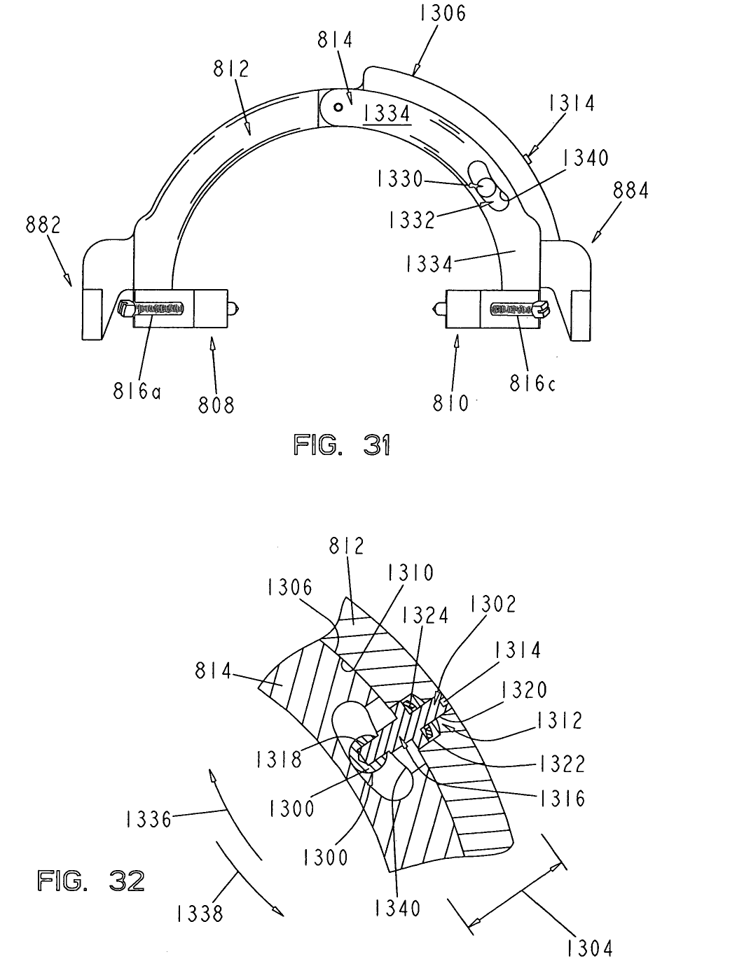

Referring to FIGS. 30 and 31, first member 1300 of force actuator 850′ is a spherical ball having a threaded aperture 1330 sized to threadably receive threaded portion 1318 of second member 1302. Ball 1300 is received in an aperture 1332 in fourth link 814 formed in a surface 1334 of fourth link 814. Aperture 1332 intersects with aperture 1312, such that threaded portion 1318 of second member 1302 may be threaded into threaded aperture 1330 while ball 1330 is positioned in aperture 1332 of fourth link 814. The intersection between apertures 1332 and 1312 is sized such that ball 1330 may not completely pass into aperture 1312. Aperture 1332 further includes a surface 1340 which bounds the intersection of apertures 1332 and 1312 and blocks the egress of ball 1330 from aperture 1332.

The force exerted by force actuator 850′ is increased by rotating screw 1302 in one of directions 1342, 1344 along its axis 1344 such that the threads of threaded portion 1318 are retracted at least partially from threaded aperture 1330 of ball 1300 and length 1304 is increased. Since shoulder portion 1316 and beveled washer 1324 of screw 1302 are larger than first portion 1320 of aperture 1312, the increase in length 1304 results in fourth link 814 being rotated in direction 834 relative to third link 812 and in ball 1330 migrating generally in a direction 1336 of aperture 1332 in fourth link 814. The force exerted by force actuator 850′ is decreased by rotating screw 1302 in the other of directions 1342, 1344 along its axis 1344 such that the threads of threaded portion 1318 are advanced further into threaded aperture 1330 of ball 1300 and length 1304 is decreased. The decrease in length 1304 results in fourth link 814 being rotated in direction 832 relative to third link 812 and in ball 1330 migrating generally in a direction 1338 of aperture 1332 in fourth link 814.

Head restraint system 800′ is applied to a head of the person as follows. In a first exemplary method, pins 816a, 816b, 816c, 816d are assembled to respective links 808, 810 such that pins 816a, 816b, 816c, 816d extend inward from links 808 and 810 by a predetermined amount. Head restraint member 800′ is placed over head 10. Fourth link 814 is gradually rotated in direction 834 such that pins 816a, 816b, 816c, 816d are brought into engagement with head 10. Fourth link 814 is rotated in direction 834 by increasing length 1304 of force actuator 850′. The increasing of length 1304 results in all four constraints 816a, 816b, 816c, 816d being simultaneously engaged with the head. At this point, head restraint member 800′ is applied to head 10. The applied head restraint member 800′ is further assembled to a body restraint device, such as body restraint member 205 to limit the movement of head 10 relative to the torso 12 of the person.

It should be noted that the force exerted by pins 816a, 816b, 816c, 816d is governed by the force exerted by force actuator 850′ on third link 812 and fourth link 814. In the illustrated example, the force exerted by pins 816a, 816b, 816c, 816d is governed by length 1304 of force actuator 850′. As such, the orthopedic surgeon may adjust the force exerted by all pins 816a, 816b, 816c, 816d by adjusting the length of force actuator 850′.

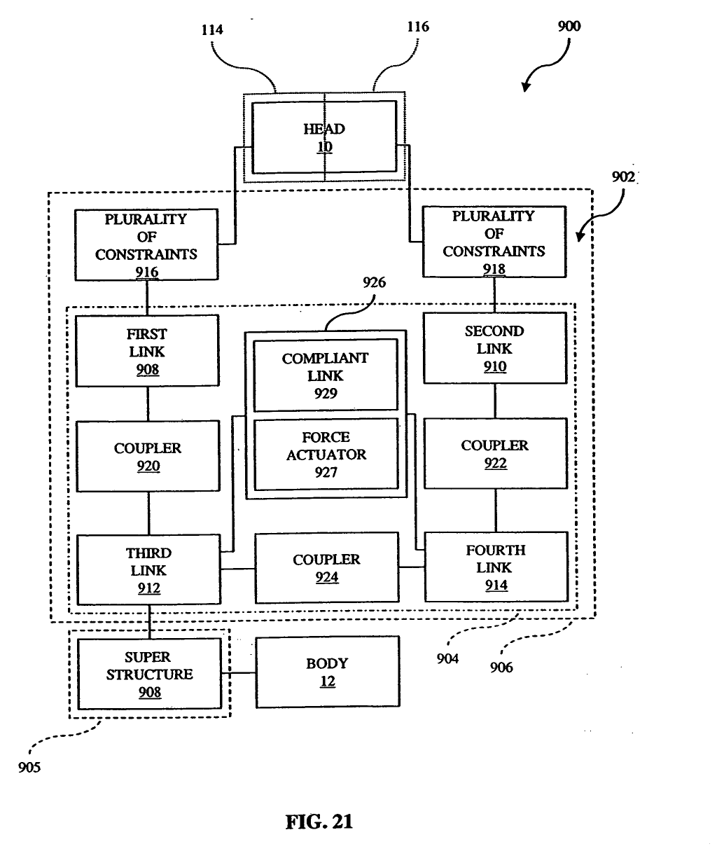

Referring to FIG. 21, an exemplary embodiment 900 of a restraint system according to the present invention is shown. Restraint system 900 includes a head restraint member 906 and a body restraint member 905. Restraint system 900 illustrates an exemplary kinematic structure 902 for an adaptive link system 904. Head restraint member 906 includes link system 904, a first plurality of constraints 916, and a second plurality of constraints 918. As discussed above in connection with FIG. 1, an adaptive link system, such as adaptive link system 904, is defined as a system of a plurality of links which is capable of automatically adapting to small changes in the geometry of head 10 once head restraint member 906 is assembled to head 10 such that head 10 remains generally fixed relative to head restraint member 906.

Link system 904 of head restraint member 906 includes a first link 908, a second link 910, a third link 912 and a four link 914. First link 908 and second link 910 are each capable of supporting a plurality of constraints 916, 918, respectively. Preferably, constraints 916, 918 are pins which are rigidly secured to the respective link of first link 908 and second link 910 such that the pins or constraints 916, 918 do not move relative to link system 904 during the time head restraint member 906 is applied to head 10. In one example, constraints 916, 918 are permanently rigidly coupled to link system 904 of head restraint member 906 by welding, gluing, epoxying, mechanical fastening, or other suitable means for permanently rigidly coupling constraints 916, 918 to head restraint member 906. In one variation, constraints 916, 918 and the respective portions of link system 904 are integrally formed or made as a single component.

In another example, each pin or constraint 916, 918 is moveably coupled to the link system 904 during the application of head restraint member 906 to head 10 and rigidly coupled to the link system 904 such that the pin or constraint 916, 918 does not move relative to link system 904 once head restraint member 906 is applied to head 10. For example, pins 916, 918 may be threadably received in respective apertures of first link 908 and second link 910 such that pins 916, 918 are moveable relative to link system 904. Pins 916, 918 are then rigidly coupled to link system 904 with a lock nut.

First link 908 is further coupled to third link 912 through a coupler 920 forming a joint between first link 908 and third link 912. Second link 910 is further coupled to fourth link 914 through a coupler 922 forming a joint between second link 910 and fourth link 914. Preferably, both of the joints formed by couplers 920 and 922 permit generally only a single degree of motion between the respective links of the joint. As such, first link 908 is capable of moving in only one degree of freedom relative to third link 912 and second link 910 is capable of moving in only one degree of freedom relative to fourth link 914. In one example, joints 920 and 922 are revolute joints. In alternative examples, the joints are one of prismatic joints or slider joints.

Third link 912 is further coupled to fourth link 914 through a coupler 924 forming a joint between third link 912 and fourth link 914. Preferably, the joint formed by coupler 924 permits generally only a single degree of motion between the respective links 912, 914. As such, fourth link 914 is capable of moving in only one degree of freedom relative to third link 912. In one example, the joint formed by coupler 924 is a revolute joint. In alternative examples, the joint are one of a prismatic joint or a slider joint.

A compliant device 926 is coupled to third link 912 and fourth link 914. Compliant device 926 exerts a force on links 912, 914 which in turn causes constraints 916, 918 to remain in contact with head 10. As such, compliant device 926 has a relaxed stated and an unrelaxed state. Compliant device 926 is coupled to third link 912 and fourth link 914 such that when link system 904 is assembled to head 10 compliant device 926 is in the unrelaxed state.

Compliant device 926 includes a force actuator 927, similar to force actuator 730 of FIG. 19, and a compliant link 929. In one embodiment, force actuator 927 exerts at least one of a tension force or a compressive force on links 912, 914. In one example, force actuator 927 is a turnbuckle which exerts a tension force on links 912, 914 and compliant link 929 is an elastic band or leaf spring. In another example, force actuator 927 is a turnbuckle which exerts a tension force on links 912, 914 and compliant link 929 is a spring such as a coil spring. In a further example, force actuator 927 is a set screw which exerts a compressive force on links 912, 914 and compliant link 929 is a spring such as a coil spring. In one embodiment, force actuator 927 is coupled to compliant link 929.

In one example, complaint device 926 is made from a non-magnetic material such that complaint device 926 does not interfere with MRI imaging of head 10. One example non-magnetic material is a glass-filled epoxy available from Composiflex located at 8100 Hawthorne Drive Erie Pa. 16509. In another example, compliant device 926 is made from any material which is capable of exerting a force on third link 912 and fourth link 914.

Compliant device 926 in combination with the joints formed by couplers 920, 922, and 924 permit link system 904 to adapt to small changes in the geometry of head 10. For instance, if the portion of head 10 engaged by one of the plurality of constraints 916 recesses, the force applied by compliant device 926 causes the relative arrangement of at least some of links 908, 910, 912, 914 to adjust such that the one constraint proximate to the recess maintains engagement with head 10. Further, in one example, compliant device 926 and the joints formed by couplers 920, 922, 924 cause the relative arrangement of at least some of links 908, 910, 912, 914 to adjust such that the one constraint proximate to the recess maintains engagement with head 10 and that all of constraints 916, 918 exert a generally uniform force against head 10. In a preferred example, compliant device 926 and the joints formed by couplers 920, 922, 924 cause the relative arrangement of at least some of links 908, 910, 912, 914 to adjust such that the one constraint proximate to the recess maintains engagement with head 10, each constraint 916, 918 exerts a generally uniform force against head 10, and each constraint 916, 918 continues to exert generally the same amount of force against head 10 as prior to the recess of head 10 occurring.

Force actuator 927 preferably orients fourth link 914 relative to third link 912. In one example, fourth link 914 is rotatably coupled to third link 912. Force actuator 927 controls the orientation of fourth link 914 relative to third link 912, such that fourth link 914 is not moveably relative to third link 912, except for movement due to compliant link 929, without a change in the force exerted by force actuator 927. By increasing the force exerted by force actuator 927 the force exerted by constraints 916, 918 against head 10 may be increased. By decreasing the force exerted by force actuator 927 the force exerted by constraints 916, 918 against head 10 may be decreased. Further, due to the characteristics of the joints formed by couplers 920, 922, 924 (only a single degree of freedom) the force exerted by each constraint 916, 918 is generally uniform compared to the remaining constraints 916, 918 and any increase or decrease in force applied by constraints 916, 918 as the result of a change in the amount of force applied by force actuator 927 is generally equal for each constraint 916, 918 compared to the remaining constraints 916, 918.

The combination of force actuator 927 and compliant link 929 in addition to characteristics of the joints formed by couplers 920, 922, 924 allow the simultaneous increasing or decreasing of force exerted by constraint 916, 918, to adapt to small changes in the geometry of head 10, to ensure that each constraint 916, 918 exerts generally the same amount of force against the head, and in the absence of a change in force of force actuator 927 that each constraint 916, 918 continues to exert generally the same amount of force against head 10 as prior to a change in the geometry of the head 10.

Head restraint member 906 when applied to head 10 should exert sufficient force to limit the movement of head 10 relative to head restraint member 906. In one example a force of at least about 30 pounds should be exerted by head restraint member 906. In another example, a force of at least about 40 pounds should be exerted by head restraint member 906. In a further example, a force of at least about 50 pounds should be exerted by head restraint member 906. In yet a further example, a force of at least about 60 pounds should be exerted by head restraint member 906. In yet another example, a force of between about 30 pounds to about 100 pounds should be executed by head restraint member 906. In still another example, a force of between about 40 pounds to about 100 pounds should be executed by head restraint member 906. In yet still another example, a force of between about 50 pounds to about 100 pounds should be executed by head restraint member 906. In still a further example, a force of between about 50 pounds to about 60 pounds should be executed by head restraint member 906.

Referring to FIG. 21, body restraint member of 905 is secured or engaged to body 12 of the person. In a preferred example, body restraint member 905 is secured to the torso of the person. Body restraint member 905 and head restraint member 906 are coupled together to limit the movement of head 10 relative to body or torso 12. In the illustrated embodiment, a superstructure 908 of body restraint member 905 is coupled to third link 912 of link system 904 of head restraint member 906. In one example, each of the exemplary superstructures described above in connection with restraint system or apparatus 100 may be coupled to head restraint member 906 to couple head 10 to torso 12.

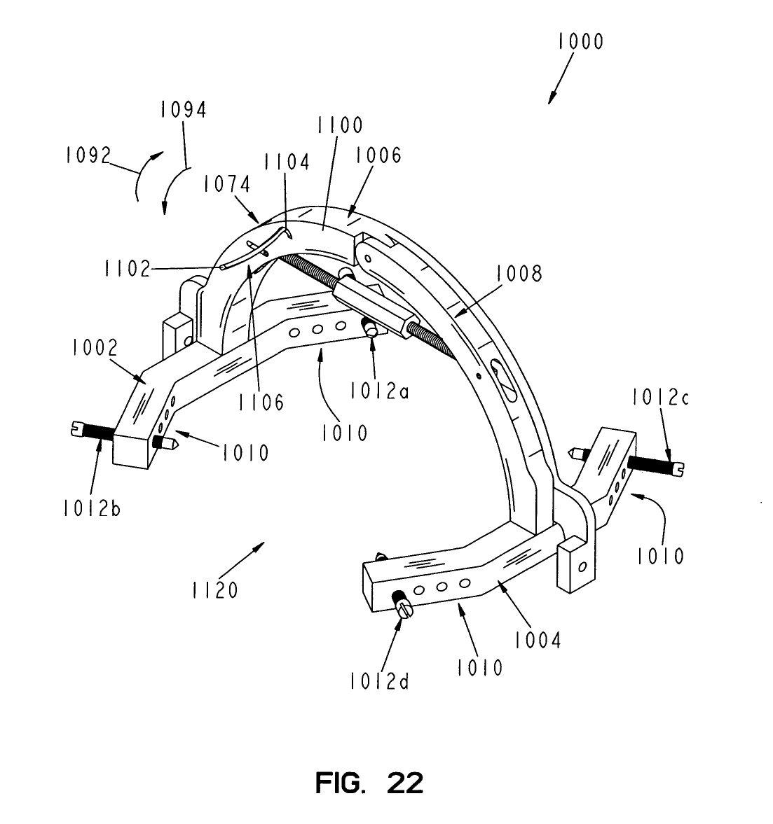

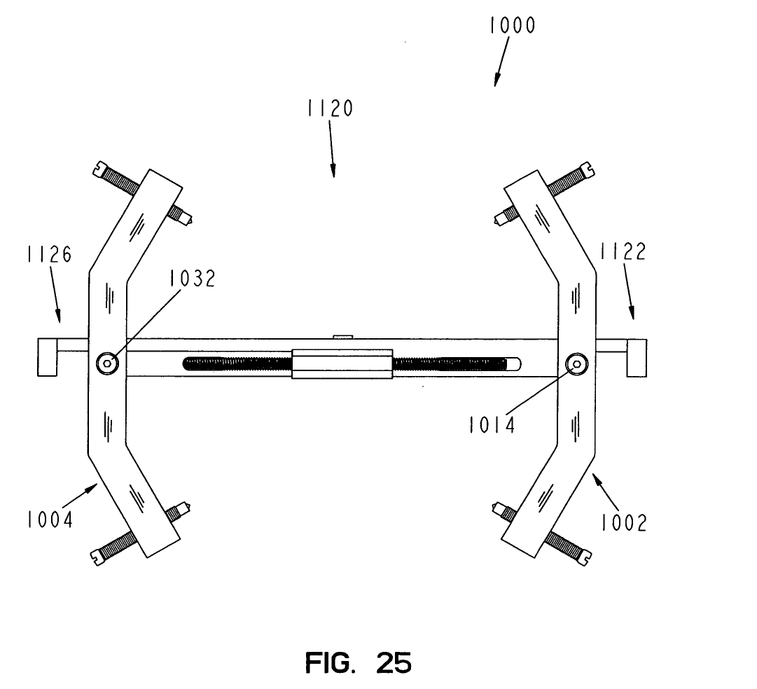

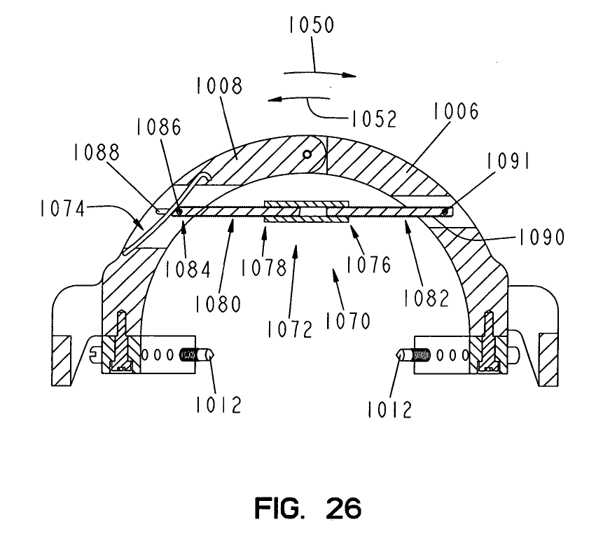

Referring to FIGS. 22-26, an exemplary head restraint system 1000 according to the present invention is shown. Head restraint member 1000 has the same kinematic structure 902 of head restraint member 906 shown in FIG. 21. Head restraint system 1000 includes a link system 1001 and constraints 1012. Link system 1001 includes a first link 1002, a second link 1004, a third link 1006, and a fourth link 1008.

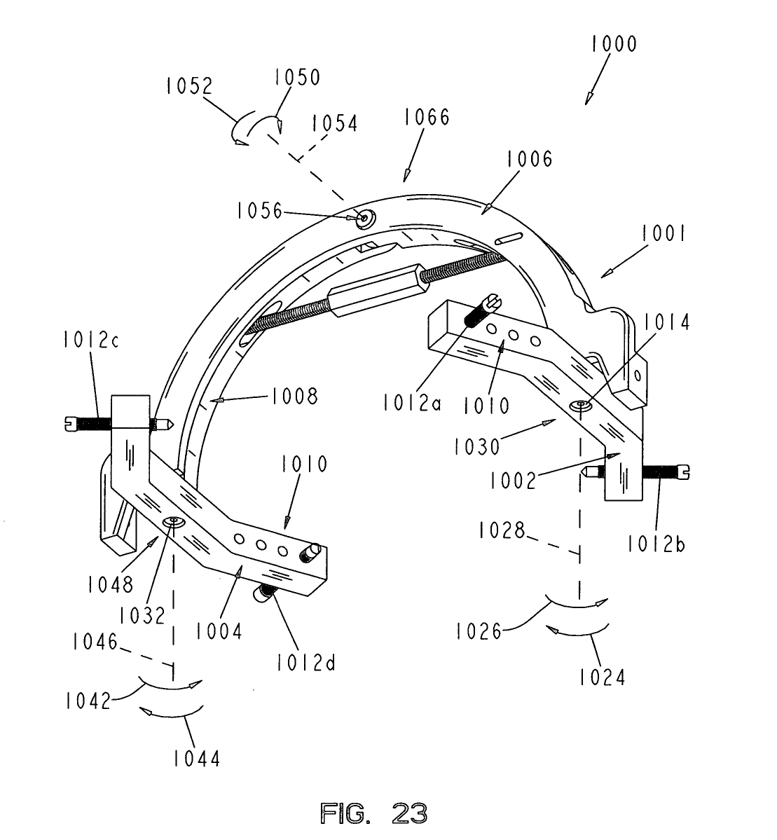

First link 1002 and second link 1004 each include a plurality of apertures 1010 to receive a plurality of constraints or pins 1012. As illustrated in FIGS. 22 and 23, first link 1002 includes plurality of apertures 1010 each configured to receive constraint 1012. First link 1002, in one example, includes two constraints 1012a and 1012b received in two of apertures 1010. It is contemplated that constraints 1012a, 1012b may be received in other apertures 1010 and/or that additional constraints 1012 are received in additional apertures 1010. Second link 1004 includes plurality of apertures 1010 each configured to receive constraint 1012. Second link 404, in one example, includes two constraints 1012c and 1012d received in two of apertures 1010. It is contemplated that constraints 1010c, 1010d may be received in other apertures 1010 and/or that additional constraints 1012 are received in additional apertures 1010.

Preferably, constraints 1012a, 1012b, 1012c, 1012d are pins which are rigidly secured to the respective link of first link 1002 and second link 1004 such that the pins or constraints 1012a, 1012b, 1012c, 1012d do not move relative to the respective link of first link 1002 and second link 1004 once head restraint member 1000 is applied to head 10. In one example, constraints 1012a, 1012b, 1012c, 1012d are permanently rigidly coupled to link system 1001 of head restraint member 1000 by welding, gluing, epoxying, mechanical fastening, or other suitable means for permanently rigidly coupling constraints 1012a, 1012b, 1012c, 1012d to head restraint member 1000. In one variation, constraints 1012a, 1012b, 1012c, 1012d and the respective first link 1002 and second link 1004 are integrally formed or made as a single component.

In another example, each pin or constraint 1012a, 1012b, 1012c, 1012d is moveably coupled to the respective link of first link 1002 and second link 1004 during the application of head restraint member 1000 to head 10 and rigidly coupled to the respective link of first link 1002 and second link 1004 such that the respective pin or constraint 1012a, 1012b, 1012c, 1012d does not move relative to the respective link of first link 1002 and second link 1004 once head restraint member 1000 is applied to head 10. For example, pins 1012a, 1012b, 1012c, 1012d may be threadably received in respective apertures of first link 1002 and second link 1004 such that pins 1012a, 1012b, 1012c, 1012d are moveable relative to link system 1004. Pins 1012a, 1012b, 1012c, 1012d are then rigidly coupled to link system 1001 with a lock nut.

Both of first link 1002 and second link 1004 include a faceted longitudinal extent approximating an arcuate longitudinal extent which in turn is an approximation of the curvature of the left half of the head and the right half of the head, respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 1002 and second link 1004 and/or orientation of apertures 1010 is such that pins or constraints 1012a, 1012b, 1012c, 1012d are generally normal to head 10 when head restraint member 1000 is applied to the head.

First link 1002 is further coupled to third link 1006 by a coupler or pin 1014 forming a joint between first link 1002 and third link 1006. Pin 1014 includes a shoulder portion and a threaded portion. First link 1002 includes an aperture sized to receive the shoulder portion of coupler 1014. Third link 1006 includes a threaded aperture which is sized to threadably receive the threaded portion of coupler 1014. Once first link 1002 is coupled to third link 1006 by coupler 1014, first link 1002 is constrained to move in a single degree of freedom, namely to rotate in directions 1024 and 1026 about an axis 1028 of coupler 1014. As such, coupler 1014 creates a revolute joint 1030 between first link 1002 and third link 1006. In alternative examples, the joint between the first link and the third link is one of a prismatic joint or a slider joint.

Second link 1004 is further coupled to fourth link 1008 by a coupler or pin 1032 formerly a joint between second link 1004 and fourth link 1008. Pin 1032 includes a shoulder portion and a threaded portion. Second link 1004 includes an aperture sized to receive the shoulder portion of coupler 1032. Fourth link 1008 includes a threaded aperture which is sized to threadably receive the threaded portion of coupler 1032. Once second link 1004 is coupled to fourth link 1008 by coupler 1032, second link 1004 is constrained to move in a single degree of freedom, namely to rotate in directions 1042 and 1044 about an axis 1046 of coupler 1032. As such, coupler 1032 creates a revolute joint 1048 between second link 1004 and fourth link 1008. In alternative examples, the joint between the second link and the fourth link is one of a prismatic joint or a slider joint.

Third link 1006 and fourth link 1008 are coupled together by a coupler 1056 such that fourth link 1008 is constrained to move in a single degree of freedom, namely to rotate in directions 1050 and 1052 about an axis 1054 of a coupler 1056. Referring to FIG. 10, coupler 1056 includes a shoulder portion and a threaded portion. Third link 1006 includes an aperture sized to receive the shoulder portion of coupler 1056. Fourth link 1008 includes a threaded aperture which is sized to threadably receive the threaded portion of coupler 1056. Once fourth link 1008 is coupled to third link 1006 by coupler 1056, fourth link 1008 is constrained to rotate in directions 1050 and 1052. As such, coupler 1056 creates a revolute joint 1066 between third link 1006 and fourth link 1008. In alternative examples, the joint between third link 1006 and fourth link 1008 is one of a prismatic joint or a slider joint.

The compliant device 1070 is coupled to third link 1010 and fourth link 1008. The compliant device 1070 exerts a force on links 1008, 1010 which in turn causes constraints 1012a, 1012b, 1012c, 1012d to remain in contact with head 10. As such, compliant device 1070 has a relaxed state and an unrelaxed state. Complaint device 1070 is coupled to third link 1010 and fourth link 1008 such that when link system 1001 is applied to head 10, compliant device 1070 is in the unrelaxed state.

Complaint device 1070 includes a force actuator 1072, similar to force actuator 927 of FIG. 19, and a compliant link 1074. In one embodiment, force actuator or force applier 1072 applies or exerts at least one of a tension force or a compressive force on links 1006, 1008. In one example, force actuator 1072 is a turnbuckle and compliant link 1074 is an elastic band or leaf spring. In another example, force actuator 1072 is a turnbuckle and compliant link 1074 is a spring such as a coil spring. In one embodiment, force actuator 1072 is coupled to compliant link 1074.

In one example, compliant device 1070 is made from a non magnetic material such that complaint device 1070 does not interfere with MRI imaging of head 10. One example, non magnetic material is a glass filled epoxy available from Composiflex located at 8100 Hawthorne Drive Erie Pa. 16509. In another example, compliant device 1070 is made from any material which is capable of exerting a force on third link 1010 and fourth link 1008.

Force actuator 1072, in the illustrated embodiment exerts a tension force on links 1010, 1008 and is a turnbuckle 1076. Turnbuckle 1076 includes a central component 1078 which is internally threaded and a pair of threaded rods 1080 and 1082. Threaded rod 1080 is rigidly coupled to compliant link 1074 at a first end 1084 and is further translatably coupled to third link 1006. A pin 1086 of threaded rod 1080 is slidably coupled to a slot 1088 and third link 1006. Threaded rod 1082 is rotatably coupled to third link 1006 at a first end 1090. Rods 1080, 1082 are further threadably received by central component 1078. By rotating central component 1078 in one of directions 1092, 1094 (see FIG. 22), rods 1080, 1082 are further advanced within central component 1078. The advancement of rods 1080, 1082 within central component 1078 results in the separation of pins 1086, 1091 being reduced and in turn fourth link 1008 is rotated in direction 1052 such that second link 1004 moves toward first link 1002. Further, the advancement of rods 1080, 1082 within central component 1078 decreases the amount of force exerted by compliant link 1074. By rotating central component 1078 in the other direction of directions 1092, 1094, rods 1080, 1082 are further retracted from within central component 1078. The retraction of rods 1080, 1082 from within central component 1078 results in the separation of pins 1086, 1091 being increased and in turn fourth link 1008 is rotated in direction 1050 such that second link 1004 moves away from first link 1002. Further, the retraction of rods 1080, 1082 from within central component 1078 results in an increase of the amount of force exerted by compliant link 1074.

Compliant link 1074 is coupled to third link 1006 adjacent surface 1100. A first end 1102 and second end 1004 of compliant link 1074 is rigidly coupled to third link 1006. A central portion 1106 of compliant link 1074 is rigidly coupled to pin 1086 of force actuator 1072. The amount of force exerted by compliant link 1074, i.e. the amount of deflection of compliant link 1074 from its relaxed state, may be adjusted by adjusting an overall length of force actuator 1072 defined by the separation between pins 1086, 1091.

Force actuator 1072 preferably orients fourth link 1008 relative to third link 1006. Force actuator 1072 controls the orientation of fourth link 1008 relative to third link 1006 such that fourth link 1008 is not moveable relative to third link 1006, except for movement due to the force exerted by compliant link 1074, without a change in the force exerted by force actuator 1072. By increasing the tension exerted by force actuator 1078 the force exerted by constraints 1012a, 1012b, 1012c, 1012d against head 10 may be increased. By decreasing the force exerted by force actuator 1072, the force exerted by constraints 1012a, 1012b, 1012c, 1012d against head 10 may be decreased. Further, due to the characteristics of the joints formed by couplers 1014, 1032, 1056 (only a single degree of freedom), the force exerted by each constraint 1012a, 1012b, 1012c, 1012d is generally uniform compared to the remaining constraints 1012a, 1012b, 1012c, 1012d in any increase or decrease in force applied by the constraints 1012 as a result of the change and the amount of force applied by force actuator 1072 is generally equal for each constraint 1012 compared to the remaining constraints 1012.

The combination of force actuator 1072 and compliant link 1074 in addition to the characteristics of the joints formed by couplers 1014, 1032, 1056 allow the simultaneous increasing or decreasing of force exerted by constraints 1012, to adapt to small changes in geometry of the head, to insure that each constraint 1012 exerts generally the same amount of force against the head, and the absence of a change in force of force actuator 1072 that each constraint 1012 continues to exert generally the same amount of force against head 10 as prior to a change in geometry of head 10.

Compliant device 1070 in combination with the joints formed by couplers 1014, 1032, 1056 permit link system 1001 to adapt to small changes in geometry to the head. For instance, if the portion of head 10 engaged by one of the plurality constraints 1012 recesses, the force applied by compliant device 1070 causes the relative arrangement of at least some of links 1002, 1004, 1006, 1008 to adjust such that the one constraint approximate to the recess maintains engagement with head 10. Further, in one example, compliant device 1070 in the joints formed by couplers 1014, 1032, 1056 cause the relative arrangement of at least some of links 1002, 1004, 1006, 1008 to adjust such that the one constraint approximate to the recess maintains engagement with head 10 in that all of constraints 1012a, 1012b, 1012c, 1012d exert a generally uniform force against head 10. In a preferred example, compliant device 1070 in the joints formed by couplers 1013, 1032, 1056 caused the relative arrangement of at least some of links 1002, 1004, 1006, 1008 to adjust such that the one constraint approximate to the recess maintains engagement with head 10, each constraint 1012a, 1012b, 1012c, 1012d exerts a generally uniform force against head 10, and each constraint 1012 continues to exert generally the same amount of force against head 10 prior to the recess of head 10 occurring.

It is contemplated that the placement of constraints 1012a, 1012b, 1012c, 1012d may be balanced or unbalanced. In one example, constraints 1012a and 1012b are generally equidistant from joint 1030 and constraints 1012c and 1012d are generally equidistance from joint 1048. In one variation, the distance from joint 1030 to each of constraints 1012a and 1012b is generally equal to the distance from joint 1048 to each of constraints 1012c and 1012d. In another variation, the distance from joint 1030 to each of constraints 1012a and 1012b is generally not equal to the distance from joint 1048 to each of constraints 1012c and 1012d. In another example, at least one pair of constraints 1012a and 1012b and constraints 1012c and 1012d are generally non-equidistant from the respective joint 1030 and joint 1048. In one variation, the distance from joint 1030 to each of constraints 1012a and 1012b is generally equal and the distance from joint 1048 to each of constraints 1012c and 1012d is generally not equal. In another variation, the distance from joint 1030 to each of constraints 1012a and 1012b is generally not equal and the distance from joint 1048 to each of constraints 1012c and 1012d is generally not equal.

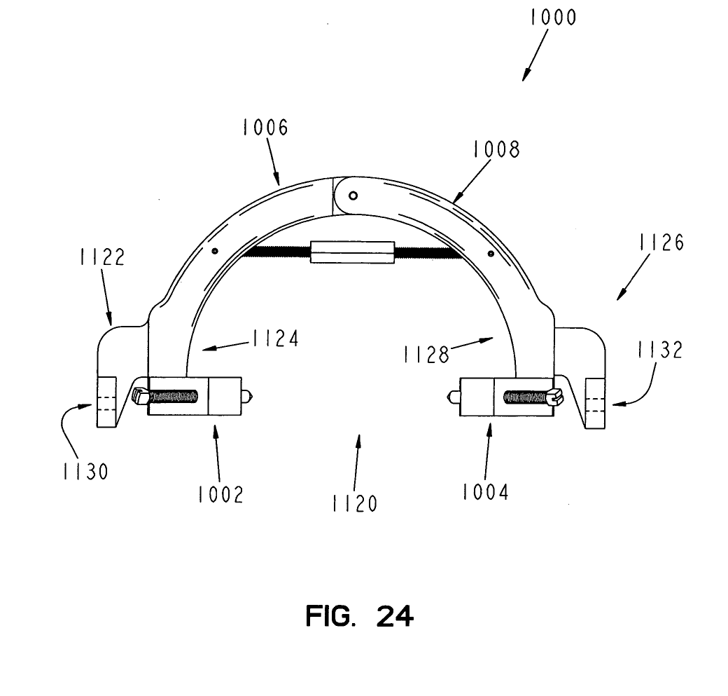

Referring to FIG. 24, third link 1006 spans an opening 1120 of head restraint member 1000 sized to receive the head of the person. Third link 1006 further includes a first mounting bracket 1122 located at first end 1124 of third link 1006 and a second mounting bracket 1126 located at a second end 1128 of third link 1006. Mounting brackets 1122, 1124 respectively include mounting apertures 1130 and 1132. Mounting apertures 1130 and 1132 are configured to receive fasteners to couple third link 1006 to a body restraint device, such as body restraint member 205 of FIG. 4. As such, third link 1006 alone provides the coupling between head restraint member 1000 and body restraint member 205. Therefore, the movement of first link 1002 relative to third link 1006, second link 1004 relative to fourth link 1008, and fourth link 1008 relative to third link 1006 is isolated from the coupling of head restraint member 1000 to body restraint member 205.

Head restraint system 1000 is applied to a head of the person as follows. In a first exemplary method, pins 1012a, 1012b, 1012c, 1012d are assembled to respective links 1002, 1004 such that pins 1012a, 1012b, 1012c, 1012d extend inward from links 1002 and 1004 by a predetermined amount. Head restraint member 1000 is placed over head 10. Force actuator 1072 is adjusted until pins 1012a, 1012b, 1012c, 1012d engage the head with sufficient force to limit movement of the head with respect to head restraint 1000. At this point, head restraint member 1000 is applied to head 10. It should be noted that when the head restraint member 1000 is applied to head 10, compliant link 1074 should be in an unrelaxed state. The applied head restraint member 1000 is further assembled to a body restraint device, such as body restraint member 205 to limit the movement of head 10 relative to the torso 12 of the person.

It should be noted that the force exerted by pins 1012a, 1012b, 1012c, 1012d is governed by the force exerted by compliant device 1070 on third link 1006 and fourth link 1008. Alternatively, in embodiments where pins 1012a, 1012b, 1012c, 1012d are threadably coupled to first link 1002 and second link 1004, the force exerted by the respective pins 1012a, 1012b, 1012c, 1012d may be adjusted by either advancing the respective pin in the respective aperture 1010 to increase the force or by retracting the respective pin outward of respective aperture 1010 to decrease the force. It should be noted that the advancement or retraction of a single constraint 1012a, 1012b, 1012c, 1012d results in a corresponding increase or decrease in force for all of constraints 1012a, 1012b, 1012c, 1012d due to the arrangement of links 1002, 1004, 1006, 1008 and compliant device 1070.

Head restraint member 1000 when applied to head 10 should exert sufficient force to limit the movement of head 10 relative to head restraint member 1000. In one example a force of at least about 30 pounds should be exerted by head restraint member 1000. In another example, a force of at least about 40 pounds should be exerted by head restraint member 1000. In a further example, a force of at least about 50 pounds should be exerted by head restraint member 1000. In yet a further example, a force of at least about 60 pounds should be exerted by head restraint member 1000. In yet another example, a force of between about 30 pounds to about 100 pounds should be executed by head restraint member 1000. In still another example, a force of between about 40 pounds to about 100 pounds should be executed by head restraint member 1000. In yet still another example, a force of between about 50 pounds to about 100 pounds should be executed by head restraint member 1000. In still a further example, a force of between about 50 pounds to about 60 pounds should be executed by head restraint member 1000.

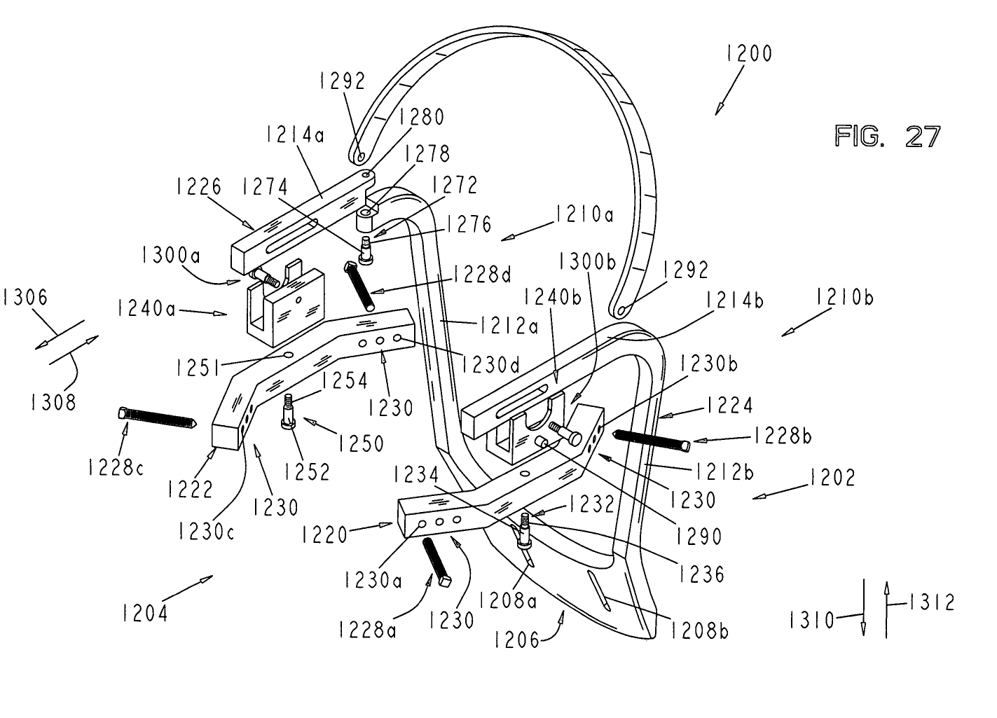

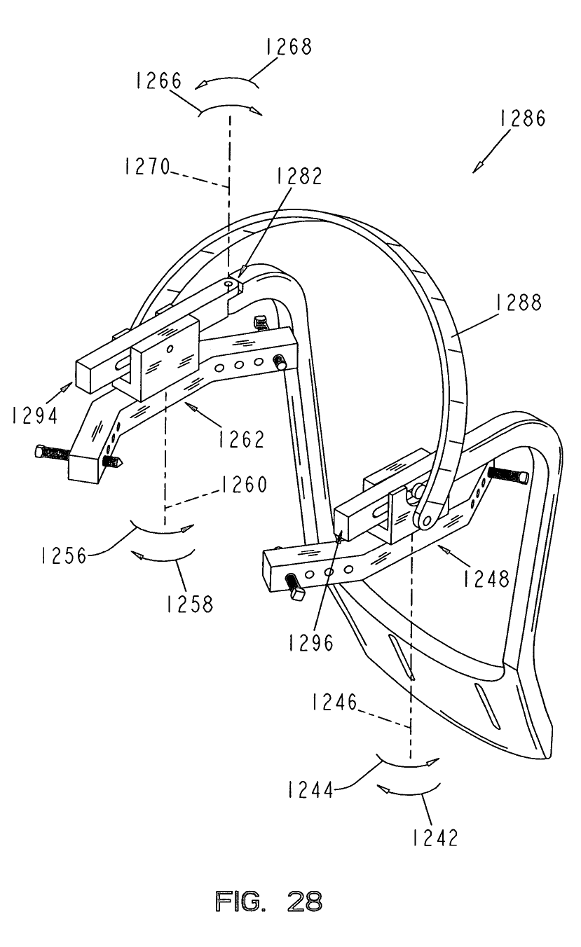

Referring to FIGS. 27 and 28, an exemplary restraint system 1200 according to the present invention is shown. Restraint system 1200 has a first portion 1202 which is a support structure and a second portion 1204 which is a head restraint member. Support structure 1202 may be used with a vest 111 (see FIG. 2) or other body restraint member 12 to couple head 10 to body 12.

Support structure 1202 includes a base member 1206 having a plurality of apertures 1208a, 1208b sized to receive fasteners such that base member 1206 may be coupled to vest 111 shown in FIG. 2 with fasteners. Support structure 1202 further includes a pair of support arms 1210a, 1210b. Each of support arms 1210a, 1210b includes a first portion 1212a, 1212b extending generally upward relative to base member 1206 and a second portion 1214a, 1214b generally angled relative to first portions 1212a, 1212b. In one example, second portions 1214a, 1214b are generally perpendicular to first portions 1212a, 1212b. In alternative examples the second portions of the support structure of FIG. 27 are angled relative to the first portions of the support structure such that an acute angle is made between the first portions and the second portions or such that an obtuse angle is made between the first portions and the second portions.

Support structure 1202 also functions as part of head restraint member 1204. Head restraint member 1204 includes a first link 1220, a second link 1222, a third link 1224, a fourth link 1226, and a plurality of constraints 1228. As shown in FIGS. 27 and 28, third link 1224 includes base member 1206, support arm 1210b, and first portion 1212a of support arm 1210a. Fourth link 1226 includes second portion 1214a of support arm 1210a.

First link 1220 and second link 1222 each include a plurality of apertures 1230 to receive a plurality of constraints or pins 1228. First link 1220 includes plurality of apertures 1230 each configured to receive constraint 1228. As shown in FIGS. 27 and 28, first link 1220, in one example, includes two constraints 1228a and 1228b received in apertures 1230a, 1230b, respectively. It is contemplated that constraints 1228a, 1228b may be received in other apertures 1230 and/or that additional constraints 1228 are received in additional apertures 1230. Second link 1222 includes plurality of apertures 1230 each configured to receive constraint 1228. As shown in FIGS. 27 and 28, second link 1222, in one example, includes two constraints 1228c and 1228d received in apertures 1230c, 1230d, respectively. It is contemplated that constraints 1228c, 1228d may be received in other apertures 1230 and/or that additional constraints 1228 are received in additional apertures 1230.

Preferably, constraints 1228a, 1228b, 1228c, 1228d are pins which are rigidly secured to the respective link of first link 1220 and second link 1222 such that the pins or constraints 1228a, 1228b, 1228c, 1228d do not move relative to the respective link of first link 1220 and second link 1222 once head restraint member 1200 is applied to head 10. In one example, constraints 1228a, 1228b, 1228c, 1228d are permanently rigidly coupled to the respective first link 1220 and second link 1222 of head restraint member 1200 by welding, gluing, epoxying, mechanical fastening, or other suitable means for permanently rigidly coupling constraints 1228a, 1228b, 1228c, 1228d to head restraint member 1200. In one variation, constraints 1228a, 1228b, 1228c, 1228d and the respective first link 1220 and second link 1222 are integrally formed or made as a single component.

In another example, each pin or constraint 1228a, 1228b, 1228c, 1228d is moveably coupled to the respective link of first link 1220 and second link 1222 during the applying of head restraint member 1200 to head 10 and rigidly coupled to the respective link of first link 1220 and second link 1222 such that the respective pin or constraint 1228a, 1228b, 1228c, 1228d does not move relative to the respective link of first link 1220 and second link 1222 once head restraint member 1200 is assembled to head 10. For example, pins 1228a, 1228b, 1228c, 1228d may be threadably received in respective apertures of first link 1220 and second link 1222 such that pins 1228a, 1228b, 1228c, 1228d are moveable relative to links 1220, 1222. Pins 1228a, 1228b, 1228c, 1228d are then rigidly coupled to links 1220 1222 with a lock nut.

Both of first link 1220 and second link 1222 include a faceted longitudinal extent approximating an arcuate longitudinal extent which in turn is an approximation of the curvature of the left half of the head and the right half of the head, respectively. In alternative embodiments, first link and second link have a circular longitudinal extent, an elliptical longitudinal extent, a parabolic longitudinal extent, a contoured longitudinal extent, or other suitable longitudinal extent to approximate the shape of head 10. Preferably, the longitudinal extent of first link 1220 and second link 1222 and/or orientation of apertures 1230 is such that pins or constraints 1228a, 1228b, 1228c, 1228d are generally normal to head 10.

First link 1220 is further coupled to a coupler 1240a of third link 1224 by a coupler or pin 1232. Coupler 1240b is described in detail below. Pin 1232 includes a shoulder portion 1234 and a threaded portion 1236. First link 1220 includes an aperture sized to receive shoulder portion 1234 of pin 1232. Coupler 1240b of third link 1224 includes a threaded aperture which is sized to threadably receive threaded portion 1236 of pin 1232. Once first link 1220 is coupled to third link 1224 by pin 1232, first link 1220 is constrained to move in a single degree of freedom, namely to rotate in directions 1242 and 1244 about an axis 1246 of pin 1232. As such, pin 1232 creates a revolute joint 1248 between first link 1220 and third link 1224. In alternative examples, the joint between first link 1220 and third link 1224 is one of a prismatic joint or a slider joint.

Second link 1222 is further coupled to a coupler 1240a of fourth link 1226 by a coupler or pin 1250. Coupler 1240a is described in detail below. Pin 1250 includes a shoulder portion 1252 and a threaded portion 1254. Second link 1222 includes an aperture 1251 sized to receive shoulder portion 1252 of pin 1250. Coupler 1240a of fourth link 1226 includes a threaded aperture which is sized to threadably receive threaded portion 1254 of pin 1250. Once second link 1222 is coupled to fourth link 1226 by pin 1250, second link 1222 is constrained to move in a single degree of freedom, namely to rotate in directions 1256 and 1258 about an axis 1260 of pin 1250. As such, pin 1250 creates a revolute joint 1262 between second link 1222 and fourth link 1226. In alternative examples, the joint between second link 1222 and fourth link 1226 is one of a prismatic joint or a slider joint.

Referring to FIGS. 27 and 28, third link 1224 and fourth link 1226 are coupled together such that fourth link 1228 is constrained to move in a single degree of freedom, namely to rotate in directions 1266 and 1268 about an axis 1270 of a coupler or pin 1272 which couples third link 1224 and fourth link 1226. Pin 1272 includes a shoulder portion 1274 and a threaded portion 1276. Third link 1224 includes an aperture 1278 sized to receive shoulder portion 1274 of pin 1272. Fourth link 1226 includes a threaded aperture 1280 which is sized to threadably receive threaded portion 1276 of pin 1272. Once fourth link 1226 is coupled to third link 1224 by pin 1272, fourth link 1226 is constrained to rotate in directions 1266 and 1268. As such, pin 1272 creates a revolute joint 1282 between third link 1224 and fourth link 1226. In alternative examples, the joint between third link 1224 and fourth link 1226 is one of a prismatic joint or a slider joint.

Although fourth link 1226 is able to rotate in both directions 1266 and 1268 relative to third link 1224, a compliant link 1286 biases fourth link 1226 in direction 1268 relative to third link 1224. In one embodiment, compliant link 1286 exerts at least on of a tension force or a compressive force. Compliant link 1286 includes an elastic band 1288 which is coupled to coupler 1240b of third link 1224 and to coupler 1240a of fourth link 1226. Couplers 1240a, 1240b each includes a pin 1290, respectively. Elastic band 1288 includes a pair of apertures 1292 which are sized to receive pins 1290. In alternative embodiments, elastic band 1288 is coupled to third link 1224 and fourth link 1226 by one or more of gluing, epoxying, mechanical fastening, or other suitable means. In an alternative embodiment, compliant member 1286 is a compression spring coupled to third link 1224 and fourth link 1226.

Elastic band 1288, in one example, is made from a non-magnetic material such that compliant member 1286 does not interfere with MRI imaging of head 10. One example non-magnetic material is a glass-filled epoxy available from Composiflex located at 8100 Hawthorne Drive Erie Pa. 16509. In another example, elastic band 1288 is made from any material which is capable of exerting a force on third link 1224 and fourth link 1226.

Compliant link 1286 may be in a relaxed state or an unrelaxed state. Compliant link 1286, in a preferred embodiment, is not in a relaxed state when head restraint member 1200 is assembled to head 10. As such, compliant link 1286 biases first end 1294 of fourth link 1226 towards first end 1296 of third link 1224 such that fourth link 1226 is biased to rotate generally in direction 1268 relative to third link 1224.

Fourth link 1226 and third link 1224 each further includes couplings 1240a, 1240b for coupling head restraint member 1204, shown in FIGS. 27 and 28. Couplings 1240a, 1240b are slidably coupled to the respective support arms 1210a, 1210b by fasteners 1300a, 1300b which are received in an elongated slot 1302a, 1302b of the respective support arms 1210a, 1210b. Fasteners 1300a, 1300b slide upon a surface 1304a, 1304b of the respective slots 1302a, 1302b and are threadably received into threaded apertures of the respective couplings 1240a, 1240b. As such, couplings 1240a, 1240b are generally restrained to move in directions 1306 and 1308 relative to support arms 1210a, 1210b. This movement allows for the positioning of first link 1220 and second link 1222 of head restraint member 1204 to be adjusted relative to support arms 1210a, 1210b. Similarly apertures 1208a, 1208b in base member 1206 are elongated such that the position of restraint 1200 is adjustable relative to the vest of the body restraint member generally in directions 1310 and 1312.

Couplings 1240a, 1240b are rigidly coupled to respective support arms 1210a, 1210b in a manner similar to the coupling of couplings 132a, 132b to support arms 126a, 126b described above in connection with FIG. 3.

In one embodiment, restraint 1200 includes a force actuator, such as a tensioning device or a compressive device. In one example, a turnbuckle is rotatably coupled to third link 1224 and fourth link 1226 proximate to first ends 1294, 1296, respectively.

It is contemplated that the placement of constraints 1228a, 1228b, 1228c, 1228d may be balanced or unbalanced. In one example, constraints 1228a and 1228b are generally equidistant from joint 1248 and constraints 1228c and 1228d are generally equidistance from joint 1262. In one variation, the distance from joint 1248 to each of constraints 1228a and 1228b is generally equal to the distance from joint 1262 to each of constraints 1228c and 1228d. In another variation, the distance from joint 1248 to each of constraints 1228a and 1228b is generally not equal to the distance from joint 1262 to each of constraints 1228c and 1228d. In another example, at least one pair of constraints 1228a and 1228b and constraints 1228c and 1228d are generally non-equidistant from the respective joint 1248 and joint 1262. In one variation, the distance from joint 1248 to each of constraints 1228a and 1228b is generally equal and the distance from joint 1262 to each of constraints 1228c and 1228d is generally not equal. In another variation, the distance from joint 1248 to each of constraints 1228a and 1228b is generally not equal and the distance from joint 1262 to each of constraints 1228c and 1228d is generally not equal.

Head restraint member 1204 when applied to head 10 should exert sufficient force to limit the movement of head 10 relative to head restraint member 1204. In one example a force of at least about 30 pounds should be exerted by head restraint member 1204. In another example, a force of at least about 40 pounds should be exerted by head restraint member 1204. In a further example, a force of at least about 50 pounds should be exerted by head restraint member 1204. In yet a further example, a force of at least about 60 pounds should be exerted by head restraint member 1204. In yet another example, a force of between about 30 pounds to about 100 pounds should be executed by head restraint member 1204. In still another example, a force of between about 40 pounds to about 100 pounds should be executed by head restraint member 1204. In yet still another example, a force of between about 50 pounds to about 100 pounds should be executed by head restraint member 1204. In still a further example, a force of between about 50 pounds to about 60 pounds should be executed by head restraint member 1204.

While the invention is susceptible to various modifications and alternative forms, exemplary embodiments thereof have been shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

We claim:

1. An apparatus for limiting the movement of the head of a person, the head having a left half and a right half either or both of which are susceptible to changes in geometry over time, the apparatus comprising:
a link system including a plurality of links;
a first plurality of constraints rigidly coupled to a first link of the link system and adapted to engage the left half of the head of the person; and
a second plurality of constraints rigidly coupled to a second link of the link system and adapted to engage the right half of the head of the person;
wherein the link system is configured to exert a force on the head of the person through the first plurality of constraints and the second plurality of constraints such that the head is generally fixed and is further configured to simultaneously adapt to changes in the geometry of the head such that the head remains generally fixed over a period of time.

2. The apparatus of claim 1, wherein the first plurality of constraints and the second plurality of constraints are pins, each pin including a pin head adapted to engage the head of the person.

3. The apparatus of claim 1, wherein the link system comprises:
a first link configured to support the first plurality of constraints;
a second link configured to support the second plurality of constraints;
a third link coupled to the first link; and
a fourth link coupled to the third link and the second link.

4. The apparatus of claim 3, wherein the first link is coupled to the third link at a first joint, the first joint configured to constrain the first link to move in a single degree of freedom relative to the third link and the second link is coupled to the fourth link at a second joint, the second joint configured to constrain the second link to move in a single degree of freedom relative to the fourth link.

5. The apparatus of claim 4, wherein the fourth link is a compliant link and is configured to provide a sufficient amount of force to engage the first plurality of constraints and the second plurality of constraints with the head of the person.

6. The apparatus of claim 5, wherein a combination of the compliant link, the first joint, and the second joint is further configured to simultaneously adapt to changes in the geometry of the head such that the head remains generally fixed relative to the head over a period of time.

7. The apparatus of claim 6, further comprising a torso restraint member adapted to be coupled to a torso of the person, the torso restraint member being coupled to the link system such that the head of the person is fixed relative to the torso of the person.

8. The apparatus of claim 4, wherein the fourth link is coupled to the third link at a third joint, the third joint configured to constrain the third link to move in a single degree of freedom relative to the fourth link.

9. The apparatus of claim 8, further comprising a compliant link coupled to the third link and the fourth link, wherein the compliant link is configured to provide a sufficient amount of force to engage the first plurality of constraints and the second plurality of constraints with the head of the person.

10. The apparatus of claim 9, wherein a combination of the compliant link, the first joint, the second joint, and the third joint is further configured to simultaneously adapt to changes in the geometry of the head such that the head remains generally fixed relative to the head over a period of time.

11. The apparatus of claim 10, further comprising a torso restraint member adapted to be coupled to a torso of the person, the torso restraint member being coupled to the link system such that the head of the person is fixed relative to the torso of the person.

12. The apparatus of claim 8, further comprising a compliant member coupled to the third link and the fourth link, the compliant member including a compliant link, wherein the compliant link is configured to provide a sufficient amount of force to engage the first plurality of constraints and the second plurality of constraints with the head of the person.

13. The apparatus of claim 12, wherein a combination of the compliant link, the first joint, the second joint, and the third joint is further configured to simultaneously adapt to changes in the geometry of the head such that the head remains generally fixed relative to the head over a period of time.

14. The apparatus of claim 13, wherein the compliant member further comprises a force applier, the force applier configured to adjust the force provided by the compliant link.

15. The apparatus of claim 14, wherein the force applier is coupled to one of the third link and the fourth link and the compliant link is coupled to the other of the third link and the fourth link, the force applier further coupled to the compliant link.

16. The apparatus of claim 14, further comprising a torso restraint member adapted to be coupled to a torso of the person, the torso restraint member being coupled to the link system such that the head of the person is fixed relative to the torso of the person.

17. An apparatus for limiting the movement of a head of a person, the apparatus comprising:
a first link configured to support a first plurality of constraints rigidly coupled to the first link and adapted to engage the left half of the head of the person;
a second link configured to support a second plurality of constraints rigidly coupled to the second link and adapted to engage the left half of the head of the person;
a third link coupled to the first link at a first joint;
a fourth link coupled to the second link at a second joint and coupled to the third link at a third joint; and
a force applier coupled to the third link and the fourth link, the force applier configured to load each of the first plurality of constraints and each of the second plurality of constraints simultaneously such that each of the first plurality of constraints and each of the second plurality of constraints engages the head with generally the same amount force.

18. The apparatus of claim 17, wherein the third link spans generally from the left half of the head at a first end of the third link to the right half of the head at a second end of the third link.

19. The apparatus of claim 17, further comprising a torso restraint member adapted to be coupled to a torso of the person, the torso restraint member being coupled to the third link such that the head of the person is fixed relative to the torso of the person.

20. A method of limiting the movement of a head of a person over time, the head being susceptible to changes in geometry over time, the method comprising the steps of:
placing a first apparatus adjacent the head of the person, the apparatus including at least a first constraint and a second constraint located adjacent a first side of the head and a third constraint and a fourth constraint located adjacent a second side of the head;
engaging each of the first, second, third, and fourth constraints with the head of the person with a force sufficient to limit the movement of the head of the person; and
automatically adapting the apparatus to changes in the geometry of the head over such that the head remains generally fixed over time.

21. The method of claim 20, wherein each of the first, second, third, and fourth constraints are engaged simultaneously.

22. The method of claim 20, further comprising the steps of:
placing a second apparatus adjacent a torso of the person, the second apparatus being secured to the torso;
coupling the first apparatus to the second apparatus such that head of the person is coupled to the torso of the person.
